(19) United States
(12) Patent Application Publication
(10) Pub. No.: US 2026/0108583 A1
(43) Pub. Date: Apr. 23, 2026

(54) COMPOSITIONS AND METHODS FOR INTRANASAL DELIVERY OF THERAPEUTIC PEPTIDES

(71) Applicant: Finitus, LLC, St. Petersburg, FL (US)

(72) Inventors: Spencer Bouhadir, Lake Worth, FL (US); Jacob J. Miguel, Pinecrest, FL (US)

(21) Appl. No.: 19/070,362

(22) Filed: Mar. 4, 2025

Related U.S. Application Data
(60) Provisional application No. 63/710,787, filed on Oct. 23, 2024.
(60) Provisional application No. 63/728,055, filed on Dec. 4, 2024.
(60) Provisional application No. 63/728,050, filed on Dec. 4, 2024.
(60) Provisional application No. 63/759,959, filed on Feb. 18, 2025.
(60) Provisional application No. 63/759,955, filed on Feb. 18, 2025.
(60) Provisional application No. 63/759,942, filed on Feb. 18, 2025.

(51) Int. Cl. A61K 38/05; A61K 9/00; A61K 9/107; A61K 47/38; A61K 47/69

(52) U.S. Cl. CPC A61K 38/05; A61K 9/0043; A61K 9/107; A61K 47/38; A61K 47/6951

(57) ABSTRACT

The present disclosure provides, inter alia, compositions and methods for intranasal delivery of peptides.

### CROSS-REFERENCE TO RELATED APPLICATIONS

The present application claims priority to and benefit of U.S. provisional application Ser. No. 63/710,787 filed Oct. 23, 2024, 63/728,055 filed Dec. 4, 2024, 63/728,050 filed Dec. 4, 2024, 63/759,959 filed Feb. 18, 2025, 63/759,955 filed Feb. 18, 2025, 63/759,942 filed Feb. 18, 2025, the entire contents of which are incorporated by reference.

### FIELD OF INVENTION

The present disclosure relates generally to intranasal delivery formulations.

### BACKGROUND OF THE INVENTION

Peptide-based therapeutics have gained significant attention in recent years due to their potential for treating a wide range of medical conditions. However, the delivery of peptides through traditional routes such as oral administration often faces challenges related to poor bioavailability and enzymatic degradation in the gastrointestinal tract. As a result, alternative delivery methods have been explored to enhance the therapeutic efficacy of peptide drugs.

Nasal drug delivery has emerged as a promising approach for administering peptides and other macromolecules. The nasal cavity offers several advantages as a site for drug absorption, including a large surface area, high vascularity, and avoidance of first-pass hepatic metabolism. Additionally, nasal administration can provide rapid onset of action and improved patient compliance compared to injectable formulations.

Despite these potential benefits, the development of stable and effective nasal spray formulations for peptides presents several technical challenges. Peptides are often susceptible to physical and chemical degradation, which can compromise their stability and therapeutic activity. Factors such as pH, temperature, and the presence of certain excipients can influence peptide stability in liquid formulations.

Furthermore, the efficient absorption of peptides across the nasal mucosa is hindered by various physiological barriers. The nasal epithelium and mucus layer can limit the permeation of large, hydrophilic molecules like peptides. Rapid mucociliary clearance in the nasal cavity also reduces the residence time of administered formulations, potentially limiting drug absorption.

To address these challenges, researchers have investigated various formulation strategies and excipients to enhance the stability and bioavailability of nasally administered peptides. These approaches may include the use of absorption enhancers, mucoadhesive agents, enzyme inhibitors, and particle engineering techniques. However, identifying compositions that are compatible and effective for delivery of peptides remains an ongoing area of research.

As the demand for non-invasive delivery methods for peptide therapeutics continues to grow, there is a need for innovative formulation approaches that can overcome the inherent challenges associated with nasal peptide delivery, such as compatibility issues between ingredients. Developing stable nasal spray compositions that maintain peptide integrity while promoting efficient absorption could potentially expand the therapeutic applications of peptide drugs and improve patient outcomes across various disease areas. The present disclosure is directed to meeting these and other needs.

### SUMMARY OF THE INVENTION

According to some aspects, the present disclosure provides an intranasal spray composition comprising: an intranasal spray formulation comprising: a peptide; a thickening agent; a cyclodextrin (CD); and a surfactant; wherein the peptide and cyclodextrin form an inclusion complex and are encapsulated by the surfactant; wherein the peptide, CMC, CD, and non-ionic surfactant are effective to produce a stable population of particles.

In some embodiments, the thickening agent is selected from the group consisting of methylcellulose, ethylcellulose, hydroxy-ethylcellulose, hydroxyl propyl cellulose, hydroxy propyl methylcellulose, carboxymethyl cellulose, sodium carboxy methylcellulose; polyacrylic acid polymers, poly hydroxyethyl methylacrylate; polyethylene oxide; polyvinyl pyrrolidone; polyvinyl alcohol, tragacanth, sodium alginate, araya gum, guar gum, xanthan gum. lectin, soluble starch, gelatin, pectin and chitosan. In some embodiments, the thickening agent is a cellulose. In some embodiments, the thickening agent is a carboxymethyl cellulose (CMC).

In some embodiments, the surfactant is a non-ionic surfactant. In some embodiments, the surfactant is selected from the group consisting of Polysorbate 80, Polysorbate 20, peg 40 hydrogenated castor oil. Surfactants also include but are not limited to: Polysorbate 80 NF, polyoxyethylene 20 sorbitan monolaurate, polyoxyethylene (4) sorbitan monolaurate, polyoxyethylene 20 sorbitan monopalmitate, polyoxyethylene 20 sorbitan monostearate, polyoxyethylene (4) sorbitan monostearate, polyoxyethylene 20 sorbitan tristearate, polyoxyethylene (5) sorbitan monooleate, polyoxyethylene 20 sorbitan trioleate, polyoxyethylene 20 sorbitan monoisostearate, sorbitan monooleate, sorbitan monolaurate, sorbitan monopalmitate, sorbitan monostearate, sorbitan trilaurate, sorbitan trioleate, sorbitan tristearate, and any combination thereof. In some embodiments, the surfactant is polysorbate 80, polysorbate 20, peg 40, hydrogenated castor oil, and combinations thereof. In some embodiments, the surfactant is

### Polysorbate 80.

In some embodiments, the cyclodextrin is selected from the group consisting of alpha cyclodextrin, beta cyclodextrin, a gamma cyclodextrin, a 2-hydroxypropyl-β-cyclodextrin, a β-cyclodextrin sulfobutylether, hydroxyethyl-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-1-β-cyclodextrin, glycosyl-β-cyclodextrin, maltosyl-β-cyclodextrin, and any combination thereof. In some embodiments, the cyclodextrin is a gamma cyclodextrin. In some embodiments, the peptide and cyclodextrin are encapsulated by the surfactant as a liposome, micelle, or noisome.

In some embodiments, the composition disclosed herein comprises 1%-40% (wt) peptide, 0.1%-5% (wt) thickening agent, 1%-15% (wt) CD, and 0.010%-0.025% surfactant. In some embodiments, the surfactant is selected from the group consisting of polysorbate 80, polysorbate 20, peg 40 hydrogenated castor oil, and combinations thereof.

In some embodiments, the composition disclosed herein comprises one or more of a preservative and a chelator. In some embodiments, the composition disclosed herein comprises one or more of potassium sorbate and EDTA. In some embodiments, the composition has a pH of 8.25 and 8.31.

In some embodiments, 90% of the particles are 50 μm or less in diameter. In some embodiments, 10% of the particles are 15 μm or less in diameter. In some embodiments, 50% of the particles are 25 μm or less in diameter.

According to some aspects, the present disclosure provides a method of treating a subject with a therapeutic formulation comprising the step of intranasally administering a composition comprising: a peptide; a thickening agent; a cyclodextrin (CD); and a surfactant; wherein the peptide and cyclodextrin form an inclusion complex and are encapsulated by the surfactant; wherein the peptide, CMC, CD, and non-ionic surfactant are effective to produce a stable population of particles.

In some embodiments, the thickening agent is selected from the group consisting of methylcellulose, ethylcellulose, hydroxy-ethylcellulose, hydroxyl propyl cellulose, hydroxy propyl methylcellulose, carboxymethyl cellulose, sodium carboxy methylcellulose; polyacrylic acid polymers, poly hydroxyethyl methylacrylate; polyethylene oxide; polyvinyl pyrrolidone; polyvinyl alcohol, tragacanth, sodium alginate, araya gum, guar gum, xanthan gum. lectin, soluble starch, gelatin, pectin and chitosan. In some embodiments, the thickening agent is a carboxymethyl cellulose (CMC).

In some embodiments, the surfactant is a non-ionic surfactant. In some embodiments, the surfactant is selected from the group consisting of Polysorbate 80, Polysorbate 20, peg 40 hydrogenated castor oil. Surfactants also include but are not limited to: Polysorbate 80 NF, polyoxyethylene 20 sorbitan monolaurate, polyoxyethylene (4) sorbitan monolaurate, polyoxyethylene 20 sorbitan monopalmitate, polyoxyethylene 20 sorbitan monostearate, polyoxyethylene (4) sorbitan monostearate, polyoxyethylene 20 sorbitan tristearate, polyoxyethylene (5) sorbitan monooleate, polyoxyethylene 20 sorbitan trioleate, polyoxyethylene 20 sorbitan monoisostearate, sorbitan monooleate, sorbitan monolaurate, sorbitan monopalmitate, sorbitan monostearate, sorbitan trilaurate, sorbitan trioleate, sorbitan tristearate, and any combination thereof. In some embodiments, the surfactant is polysorbate 80, polysorbate 20, peg 40, hydrogenated castor oil, and combinations thereof. In some embodiments, the surfactant is

### Polysorbate 80.

In some embodiments, the cyclodextrin is selected from the group consisting of alpha cyclodextrin, beta cyclodextrin, a gamma cyclodextrin, a 2-hydroxypropyl-β-cyclodextrin, a β-cyclodextrin sulfobutylether, hydroxyethyl-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-1-β-cyclodextrin, glycosyl-β-cyclodextrin, maltosyl-β-cyclodextrin, and any combination thereof. In some embodiments, the cyclodextrin is a gamma cyclodextrin.

In some embodiments, the peptide and cyclodextrin are encapsulated by the surfactant as a liposome, micelle, or noisome.

In some embodiments, the composition and/or methods disclosed herein comprise 1%-40% (wt) peptide, 0.1%-5% (wt) thickening agent, 1%-15% (wt) CD, and 0.010%-0.025% surfactant. In some embodiments, the surfactant is selected from the group consisting of polysorbate 80, polysorbate 20, peg 40 hydrogenated castor oil, and combinations thereof. In some embodiments, the composition and/or methods disclosed herein comprise one or more of a preservative and a chelator. In some embodiments, the composition and/or methods disclosed herein comprise one or more of potassium sorbate and EDTA. In some embodiments, the composition has a pH of 8.25 and 8.31.

In some embodiments, 90% of the particles are 50 μm or less in diameter. In some embodiments, 10% of the particles are 15 μm or less in diameter. In some embodiments, 50% of the particles are 25 μm or less in diameter.

According to some aspects, the present disclosure provides a method for delivery of a peptide to the central nervous system of a subject by intranasal administration comprising the step of: providing to the subject a compositions comprising a peptide; a thickening agent; a cyclodextrin (CD); and a surfactant; wherein the peptide and cyclodextrin form an inclusion complex and are encapsulated by the surfactant; wherein the peptide, CMC, CD, and non-ionic surfactant are effective to produce a stable population of particles.

In some embodiments, the composition is administered intranasally 2 or more times per day.

In some embodiments, the thickening agent is selected from the group consisting of methylcellulose, ethylcellulose, hydroxy-ethylcellulose, hydroxyl propyl cellulose, hydroxy propyl methylcellulose, carboxymethyl cellulose, sodium carboxy methylcellulose; polyacrylic acid polymers, poly hydroxyethyl methylacrylate; polyethylene oxide; polyvinyl pyrrolidone; polyvinyl alcohol, tragacanth, sodium alginate, araya gum, guar gum, xanthan gum. lectin, soluble starch, gelatin, pectin and chitosan. In some embodiments, the thickening agent is a carboxymethyl cellulose (CMC). In some embodiments, the surfactant is a non-ionic surfactant.

In some embodiments, the surfactant is selected from the group consisting of Polysorbate 80, Polysorbate 20, peg 40 hydrogenated castor oil. Surfactants also include but are not limited to: Polysorbate 80 NF, polyoxyethylene 20 sorbitan monolaurate, polyoxyethylene (4) sorbitan monolaurate, polyoxyethylene 20 sorbitan monopalmitate, polyoxyethylene 20 sorbitan monostearate, polyoxyethylene (4) sorbitan monostearate, polyoxyethylene 20 sorbitan tristearate, polyoxyethylene (5) sorbitan monooleate, polyoxyethylene 20 sorbitan trioleate, polyoxyethylene 20 sorbitan monoisostearate, sorbitan monooleate, sorbitan monolaurate, sorbitan monopalmitate, sorbitan monostearate, sorbitan trilaurate, sorbitan trioleate, sorbitan tristearate, and any combination thereof. In some embodiments, the surfactant is polysorbate 80, polysorbate 20, peg 40, hydrogenated castor oil, and combinations thereof. In some embodiments, the surfactant is

### Polysorbate 80.

In some embodiments, the cyclodextrin is selected from the group consisting of alpha cyclodextrin, beta cyclodextrin, a gamma cyclodextrin, a 2-hydroxypropyl-β-cyclodextrin, a β-cyclodextrin sulfobutylether, hydroxyethyl-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-1-β-cyclodextrin, glycosyl-β-cyclodextrin, maltosyl-β-cyclodextrin, and any combination thereof. In some embodiments, the cyclodextrin is a gamma cyclodextrin. In some embodiments, the peptide and cyclodextrin are encapsulated by the surfactant as a liposome, micelle, or noisome.

In some embodiments, the compositions and methods disclosed herein comprise 1%-40% (wt) peptide, 0.1%-5% (wt) thickening agent, 1%-15% (wt) CD, and 0.010%-0.025% surfactant. In some embodiments, the surfactant is selected from the group consisting of polysorbate 80, polysorbate 20, peg 40 hydrogenated castor oil, and combinations thereof. In some embodiments, the compositions and methods disclosed herein comprise one or more of a preservative and a chelator. In some embodiments, the compositions and methods disclosed herein comprise one or more of potassium sorbate and EDTA. In some embodiments, the composition has a pH of 8.25 and 8.31.

In some embodiments, 90% of the particles are 50 μm or less in diameter. In some embodiments, 10% of the particles are 15 μm or less in diameter. In some embodiments, 50% of the particles are 25 μm or less in diameter.

According to some aspects, the present disclosure provides an intranasal composition made by the process of: dissolving a peptide (e.g. carnosine) in water to obtain a clear solution; adding cyclodextrin under stirring to form a complex with carnosine; adding a surfactant (e.g. P80) to form a hybrid micelle and cyclodextrin complex; and adding CMC with mixing to maintain the viscosity of formulation. In some embodiments, potassium sorbate is then added. In some embodiments, EDTA is added.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIG. 1 shows a visual representation of the intranasal compositions disclosed herein.

FIG. 2 shows an HPLC Chromatograms of 1 ug/mL S212 solutions and components after storage at 40° C., including: Solution with only S212 (FIG. 2A); solution containing MCG591p (FIG. 2B, Sample 2); solution containing p80 (FIG. 2C, Sample 4); solution with potassium sorbate (FIG. 2D, Sample 6); and solution with EDTA (FIG. 2E, Sample 8); solution containing p80 (FIG. 2E/2F, Sample 4); solution with potassium sorbate (FIG. 2G/2H, Sample 6); and solution with EDTA (FIG. 2I/2J, Sample 8).

FIG. 3A to 3F show particle size measurements and statistical analysis of three samples using the SHIMADZU SALD-7001 Laser Diffraction Particle Size Analyzer.

FIG. 4 shows a histogram representation of particle size as measured by optical microscope.

FIGS. 5A and 5B shows data of changes in gene expression after treatment of carnosine in forebrain neurons (FIG. 5A) and astrocytes (FIG. 5B).

FIG. 6 shows data of the effect of S212 nasal spray via intranasal drug delivery for 10 days (once a day) on the Latency of the first fall in the Rotarod test in MPTP-induced subacute PD model mice (x±se, n=10)

FIG. 7 shows data of the effect of S212 nasal spray via intranasal drug delivery for 10 days (once a day) on the Frequency of the fall in the Rotarod test in MPTP-induced subacute PD model mice (x±se, n=10).

FIG. 8 show data of the effect of S212 nasal spray via intranasal drug delivery for 10 days (once a day) on the Time of climbing pole in MPTP-induced subacute PD model mice (x=se, n=10)

FIG. 9 shows data of the effect of S212 nasal spray via intranasal drug delivery on the gait score climbing a pole in MPTP-induced subacute PD model mice (x±se, n=10).

FIG. 10 shows data of the effect of S212 nasal spray via intranasal drug delivery on the grip strength in MPTP-induced subacute PD model mice (x±se, n=10).

FIG. 11A to 11C shows the blood collection sequence for plasma concentration of L-Carnosine in a rodent model.

FIG. 12A is data showing individual and mean plasma concentration-time data of L-carnosine. FIG. 12B is data showing the pK parameters observed and plasma concentration-time profiles of L-Carnosine.

### DETAILED DESCRIPTION

### Definitions

The term “about” is used here in conjunction with numeric values to include normal variations in measurements as expected by persons skilled in the art, and is understood have the same meaning as “approximately” and to cover a typical margin of error, such as +5% of the stated value.

Terms such as “a,” “an,” and “the” are not intended to refer to only a singular entity, but include the general class of which a specific example may be used for illustration.

The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination y two or more of the listed elements.

Any amounts (e.g., concentrations) of components in a composition given as a percentage (%) refer to a percentage by weight per volume unless otherwise indicated.

As used herein, the term “nonionic surfactant” refers to a molecule that acts as an uncharged surfactant. Surfactants are chemical compounds that decrease the surface tension or interfacial tension between two liquids, a liquid and a gas, or a liquid and a solid.

As used herein, “subject” refers to any mammal, including humans, domestic and farm animals, and zoo, sports, or pet animals, such as dogs, horses, cats, sheep, pigs, cows, etc. The preferred subject herein is a human subject, including adults, children, and the elderly.

The terms “treat,” “treated,” or “treating” as used herein refers to therapeutic treatment and/or prophylactic or preventative measures, wherein the object is to prevent or slow down (lessen) an undesired physiological condition, disorder or disease, or to obtain beneficial or desired clinical results. As used herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects.

As used herein, the term a “therapeutically effective amount” is an amount sufficient to effect beneficial or desired results. A therapeutically effective amount can be administered in one or more doses. The therapeutically effective amount is generally determined by a physician on a case-by-case basis and is within the skill of one in the art. Several factors are typically taken into account when determining an appropriate dosage. These factors include age, sex and weight of the patient, the condition being treated, the severity of the condition and the form of the composition being administered.

References to a “neurodegenerative condition”, a “neurodegenerative disorder” or a “neurodegenerative disease”, are used interchangeably, and should be understood as a reference to a condition characterized by neurologically based cognitive, emotional and behavioral disturbances. Neurodegenerative conditions may affect brain or peripheral nerve function. They result from the deterioration of neurons and they are characterized by progressive central or peripheral nervous dysfunction. They are divided into two groups: conditions causing problems with movement or sensation and conditions affecting memory or related to dementia. For example, neurodegenerative conditions in accordance with the invention may include Alzheimer's disease, Alexander disease, Alper's disease, amyotrophic lateral sclerosis, ataxia, telangiectasia, Canavan disease, Cockayne syndrome, corticobasal degeneration, Creutzfeldt-Jakob disease, dementia, Huntington disease, Kennedy's disease, Krabbe disease, Lewy body dementia, Machado-Joseph disease, multiple sclerosis, Parkinson's disease, Pelizaeus-Merzbacher disease, Fronto-Temporal Dementia, Pick's disease, primary lateral sclerosis, Refsum's disease, Sandhoff disease, Schilder's disease, Steele-Richardson-Olszewski disease, tabes dorsalis, Guillain-Barre Syndrome and peripheral neuropathies such as traumatic (nerve severing or crushing), ischemic, metabolic (diabetes, uraemia), infectious, alcoholic, iatrogenic, and genetic neuropathies.

The term “dementia”, as used herein would be clear to persons skilled in the art and includes conditions characterized by neurologically-based cognitive, emotional and behavioral impairment, in particular the Diagnostic and Statistical Manual of Mental Disorders IV outlines characterizes dementia by the presence of: Multiple cognitive deficits, including memory impairment and at least one of the following: aphasia, apraxia, agnosia or disturbance in executive functioning; and impairment of social or occupational function.

Reference to “characteristic symptoms of dementia”, “characteristic symptoms of Alzheimer's disease”, and “characteristic symptoms of Parkinson's disease” should be understood as a reference to any one or more symptoms which may occur in an individual suffering from dementia, in particular dementia associated with disease such as Alzheimer's disease or Parkinson's disease. These symptoms may be evident throughout the disease course or they may be evident only transiently or periodically. For example, an individual may exhibit severe memory impairment impaired social function in response to specific environmental cues or stressors. It should also be understood that the subject symptoms may not necessarily be exhibited by all individuals suffering from Alzheimer's disease or Parkinson's disease. For example, some individuals may suffer from cognitive deficits without obvious impairment of social function. However, for the purpose of the present invention, any such symptoms, irrespective of how many or few patients ever actually exhibit the given symptom, are encompassed by this definition. Without limiting the present invention to any one theory or mode of action, the symptoms that are most commonly associated with Alzheimer's disease include cognitive deficits and impaired social or occupational function, and the symptoms that are most commonly associated with Parkinson's disease include impaired gross and fine motor functions. For certain of the abovementioned conditions it is clear that the methods of the invention may be used prophylactically as well as for the alleviation of acute symptoms.

As used herein, the term “liposome” refers to a generally spherical cluster or aggregate of amphiphilic compounds (including lipid compounds), generally in the form of one or more concentric layers (e.g., bilayers).

As used herein the term “niosome” refers to a unilamellar or multilamellar vesicle comprising non-ionic surfactants, and optionally cholesterol and a charged molecule. In some embodiments, the non-ionic surfactants comprise alkyl ethers, alkyl esters, alkyl amides, fatty acid and amino acid compounds. In some embodiments, niosomes of the instant disclosure comprises no phospholipid as a component of the membrane.

As used herein, the term “lyoprotection” refers to stabilization during all of the freeze-drying process (i.e., during both freezing and drying). Such stabilization is often required for freeze-drying of materials such as proteins, peptides and biological drugs. This is because complex molecules often require a moderate level of residual water to maintain structure and function. Accordingly, a “lyoprotectant” protects the structure and/or function of composition during lyophilization (e.g., prevents aggregation, improves bioavailability, increases stability and/or improves membrane integrity and cargo retention).

As used herein, the term “lyophilization” (also known as “lyophilizing,” “freeze drying” or “cryodessication”) refers to a low temperature dehydration process that involves freezing a product and lowering pressure, removing the ice by sublimation. Lyophilizing may comprise freezing the composition at a temperature of greater than −40° C., or e.g. 55 less than-30° C., forming a frozen composition; and drying the frozen composition to form the lyophilized composition. The step of drying may occur at 50 mTorr at a temperature of −25 to −34° C., or −30 to −34° C.

As used herein, the term “encapsulated” by a surfactant with respect to some composition disclosed herein (e.g., a peptide/cyclodextrin encapsulated by a surfactant) means that the composition is embedded within any part of a structure (e.g. micelles, liposomes, etc.) formed by the surfactant. The composition may be located in any part of the structure, such as within an inner cavity of the structure (e.g. the aqueous inner cavity of a liposome), embedded in the hydrophilic region of the structure, or embedded in the hydrophobic region of the structure.

### Intranasal Formulations

According to some aspects, present disclosure provides a nasal spray composition comprising a peptide, a thickening agent (such as a carboxymethyl cellulose (CMC)), and a surfactant. According to some aspects, the present disclosure provides a nasal spray composition comprising a peptide, a thickening agent (such as a carboxymethyl cellulose (CMC)), a cyclodextrin (CD), and a surfactant. In some embodiments, the peptide and cyclodextrin form an inclusion complex and are encapsulated by the surfactant, wherein the peptide, CMC, CD, and non-ionic surfactant are effective to produce a stable population of particles.

According to some such embodiments, this arrangement of components significantly reduces variables associated with thickening agent and CD interactions. Thickening agents, such as cellulose derivatives (e.g. CMC) often comprise large organic molecules that may have undesired characteristics when interacting with CD. For example, the combination of CMC and CD in nasal spray formulations may lead to unpredictable outcomes due to several factors. In some cases, the interaction between these two components may result in complex physicochemical behaviors that are not easily predicted based on their individual properties. CMC, a linear polymer of cellulose with carboxymethyl groups, may form a network structure in aqueous solutions. This network may contribute to the viscosity and mucoadhesive properties of the formulation. On the other hand, cyclodextrins are cyclic oligosaccharides with a hydrophobic cavity that can form inclusion complexes with various molecules, including peptides.

When combined, CMC and CD may interact in ways that result in unexpected changes to the formulation's characteristics. The unpredictability of combining CMC and CD may stem from several factors such as: 1. Competition for water molecules: Both CMC and CD have hydrophilic regions that interact with water. In some cases, this competition may affect the hydration state of the peptide or other components in the formulation. 2. Influence on peptide-cyclodextrin complexation: The presence of CMC may alter the equilibrium of peptide-cyclodextrin complex formation. In some instances, this could enhance complex stability, while in others, it may interfere with the desired inclusion. 3. Changes in rheological properties: The interaction between CMC and CD may lead to unexpected changes in the viscosity or flow behavior of the nasal spray. This could impact the spray characteristics and deposition pattern in the nasal cavity. 4. pH-dependent interactions: The ionization state of CMC may be influenced by the formulation's pH, which in turn could affect its interaction with cyclodextrins and the peptide. This pH-dependency may introduce additional variability in the formulation's behavior. 5. Impact on particle size distribution: The combination of CMC and CD may influence the formation and stability of particles or droplets in the nasal spray. In some cases, this could lead to unexpected changes in the particle size distribution, affecting the spray's performance. 6. Potential for aggregate formation: Under certain conditions, the interaction between CMC and CD may promote the formation of aggregates or complexes that could impact the stability or bioavailability of the peptide. 7. Influence on preservative efficacy: The network formed by CMC and the inclusion properties of CD may affect the distribution and activity of preservatives in the formulation, potentially leading to unexpected changes in antimicrobial efficacy. 8. Effects on absorption enhancers: If absorption enhancers are used in the formulation, the CMC-CD combination may interact with these components in ways that are not easily predicted, potentially altering their effectiveness.

Similarly, the interaction between thickening agents and peptides in nasal sprays may have undesired characteristics. For example, the interaction between CMC and peptides in nasal spray formulations may be unpredictable due to several factors, including: 1. Charge interactions: CMC is typically negatively charged, while peptides may have varying charge distributions depending on their amino acid composition and the pH of the formulation. In some cases, electrostatic attractions or repulsions between CMC and peptides may lead to unexpected complex formation or changes in peptide conformation. 2. Hydrogen bonding: Both CMC and peptides can form hydrogen bonds. In some aspects, the extent and nature of hydrogen bonding between CMC and peptides may vary depending on factors such as temperature, pH, and ionic strength of the formulation. 3. Hydrophobic interactions: Some peptides may have hydrophobic regions that can interact with the hydrophobic backbone of CMC. In certain cases, these interactions may affect the solubility or stability of the peptide in unpredictable ways. 4. Conformational changes: The presence of CMC may induce conformational changes in peptides, potentially altering their biological activity or stability. In some instances, these changes may be difficult to predict without extensive experimental studies. 5. Competitive interactions: CMC may compete with peptides for interactions with other components in the formulation, such as cyclodextrins or surfactants. This competition may lead to unexpected changes in the overall formulation properties. 6. Molecular weight and degree of substitution: The molecular weight and degree of substitution of CMC can vary, potentially leading to different interaction profiles with peptides. In some cases, these variations may result in unpredictable effects on formulation stability and performance. 7. Concentration-dependent effects: The ratio of CMC to peptide may influence the nature and extent of their interactions. In certain aspects, changes in concentration may lead to non-linear effects on formulation properties. 8. Environmental factors: Temperature, pH, and ionic strength may all influence the interaction between CMC and peptides. In some instances, small changes in these parameters may result in significant and unpredictable alterations in formulation behavior. 9. Kinetic effects: The rate at which CMC and peptides interact may vary depending on formulation conditions. In some cases, this may lead to time-dependent changes in formulation properties that are difficult to predict. 10. Synergistic effects with other excipients: The presence of other components in the nasal spray formulation may modulate the interaction between CMC and peptides in complex ways. In certain aspects, these multi-component interactions may result in emergent properties that are not easily predictable from the behavior of individual components.

Here is has been surprisingly discovered that the uncertainties of the thickening agent-CD and/or thickening agent-peptide interaction can be reduced or eliminated by encapsulating the peptide/CD in a surfactant-based particle followed by addition of thickening agent. The thickening agent is then able to effectively control viscosity of the overall solution while minimizing interaction with peptide/CD. Thus, when arranged in this way, a composition comprising peptide, thickening agent, CD, and surfactant are effective to produce a stable population of particles.

In one or more embodiments, the present disclosure provides intranasal compositions for administration to a subject. In some embodiments, the present disclosure provides intranasal compositions for treatment of neurodegenerative diseases or disorders. As one non-limiting example, it has been surprisingly found that the particles disclosed herein are effective for intranasal delivery of an antioxidant that is effective to treat a neurodegenerative disease such as Parkinson's disease. In some embodiments, intranasal delivery of a composition comprising peptide, a surfactant, and cellulose derivative may enhance delivery of the peptide to the central nervous system.

The formulations as disclosed herein may allow an active agent, such as a peptide (e.g. carnosine), to be absorbed in a sustained manner providing improved bioavailability at low or reduced doses and/or longer duration of action. In some embodiments, the formulations of the present invention may also provide a reduced incidence of side effects when compared with other drug delivery methods.

### Thickening Agent

According to some aspects, the present disclosure provides an aqueous intranasal formulation comprising peptide, a surfactant, and a thickening agent. In some embodiments, the intranasal formulation comprising carnosine, a surfactant, and thickening agent may advantageously provide a balance between ease of administration by intranasal delivery and adherence of the formulation to the nasal mucosa. In particular, the intranasal formulation disclosed herein may be administered as a stable intranasal spray yet provide sufficient residence time on the nasal mucosa to allow trans-nasal absorption of the active agent. Furthermore, the intranasal formulations disclosed herein may additionally allow for a low or reduced dose of an active agent to be administered, sustained release of the active agent, longer duration of action, and/or a reduced incidence of side effects when compared with other delivery methods.

The thickening agent of the intranasal formulations disclosed herein may modify the viscosity of the formulation to provide improved adherence of the formulation to the nasal mucosa without adversely affecting the ease of administration, in particular administration as an intranasal spray. Without wishing to be bound by theory, the thickening agent may additionally increase the residence time of the formulation on the nasal mucosa, reduce loss of the formulation via mucociliary clearance of the nasal passages and/or improve the trans-nasal absorption. In some embodiments, the thickening agent may comprise about 0.1% to about 10% by weight of the total composition.

In some embodiments, the thickening agent as disclosed herein may be any pharmaceutically acceptable, nasal mucosa-tolerant excipient known to those skilled in the art. The thickening agent in as disclosed herein may advantageously contribute to the controlled release of the active ingredient on the mucosal membranes. Suitable thickening agents in accordance with the invention include methylcellulose, ethylcellulose, hydroxy-ethylcellulose, hydroxyl propyl cellulose, hydroxy propyl methylcellulose, carboxymethyl cellulose, sodium carboxy methylcellulose; polyacrylic acid polymers, poly hydroxyethyl methylacrylate; polyethylene oxide; polyvinyl pyrrolidone; polyvinyl alcohol, tragacanth, sodium alginate, araya gum, guar gum, xanthan gum. lectin, soluble starch, gelatin, pectin and chitosan.

According to some embodiments, the intranasal composition disclosed herein comprises an amount of a thickening agent which improves adherence of the formulation to the nasal mucosa without adversely affecting administration of the formulation as an intranasal spray. In one or more embodiments, the thickening agent may comprise about 0.1% to about 4% by weight of the total composition, about 0.25% to about 2% by weight of the total composition, or about 0.5% to about 1% by weight of the total composition.

In some embodiments, the intranasal composition disclosed herein comprises one or more membrane penetration-enhancing agents such as (i) a surfactant, (ii) a bile salt, (iii) a phospholipid or fatty acid additive, micelle, liposome, or carrier, (iv) an alcohol, (v) an enamine, (iv) an NO donor compound, (vii) a long-chain amphipathic molecule (viii) a small hydrophobic penetration enhancer; (ix) sodium or a salicylic acid derivative; (x) a glycerol ester of acetoacetic acid (xi) a cyclodextrin or beta-cyclodextrin derivative, (xii) a medium-chain fatty acid, (xiii) a chelating agent, (xiv) an amino acid or salt thereof, (xv) an N-acetylamino acid or salt thereof, (xvi) an enzyme degradative to a selected membrane component, (xvii) an inhibitor of fatty acid synthesis; or any combination thereof.

In some embodiments, the intranasal composition comprises a cellulose derivative. In some embodiments, the cellulose derivative is one or more of cellulose esters, organic ester (such as Cellulose acetate, Cellulose triacetate, Cellulose propionate, Cellulose acetate propionate (CAP), Cellulose acetate butyrate (CAB)), inorganic esters (such as Cellulose nitrate and Cellulose sulfate), cellulose ethers, alkyl derivatives (such as Methylcellulose, Ethylcellulose, and Methyl-ethyl cellulose), hydroxyalkyl derivatives (such as Hydroxy-ethyl cellulose, Hydroxypropyl cellulose, Hydroxy ethyl-methyl cellulose, Hydroxypropyl methylcellulose, and Ethyl hydroxyethyl cellulose), and carboxyalkyl derivatives (such as Carboxymethyl cellulose (CMC)). In some embodiments, the intranasal composition comprises hydrophilic polymers such as hyaluronic acid.

In some embodiments, the intranasal composition comprises, by weight of the total composition 0.1%-5% (such as 0.5, 0.7, 1, 1.5, 2, 2.5, 3, 3.5, 4, 4.5, 5% or any value therebetween) cellulose derivative. In some embodiments, the intranasal composition comprises, by weight of the total composition 0.1%-5% (such as 0.5, 0.7, 1, 1.5, 2, 2.5, 3, 3.5, 4, 4.5, 5% or any value therebetween) carboxymethyl cellulose (CMC).

### Peptides

In some embodiments, the intranasal composition comprises a peptide. In some embodiments, the intranasal composition comprises an antioxidant peptide. In some embodiments, the intranasal composition comprises an amino acid, peptide, or peptoid with a molecular weight of 65 g/mol to 800 g/mol. In some embodiments, the intranasal composition comprises an amino acid, peptide, or peptoid with an isoelectric point of 7 to 10 pI. In some embodiments, the intranasal composition comprises an amino acid, peptide, or peptoid with a hydrophobicity of +8 Kcal*mol −1 to 15 Kcal*mol-1.

In some embodiments, the intranasal composition comprises one or more of: Carnosine (β-Ala-His), Anserine (β-Ala-1-methylHis), Kyotorphin (Tyr-Arg), Thyrotropin-releasing hormone (pGlu-His-Pro), Glycylglycine (Gly-Gly), Aspartame (Asp-Phe-OMe), Glutathione (γ-Glu-Cys-Gly), RGD (Arg-Gly-Asp), Tuftsin (Thr-Lys-Pro-Arg), Melanostatin (Pro-Leu-Gly), Glycyl-L-glutamine (Gly-Gln), L-alanyl-L-glutamine (Ala-Gln), Seryltyrosine (Ser-Tyr), Prolylleucylglycine amide (Pro-Leu-Gly-NH2), Tyr-MIF-1 (Tyr-Pro-Leu-Gly-NH2), Endomorphin-1 (Tyr-Pro-Trp-Phe-NH2), Endomorphin-2 (Tyr-Pro-Phe-Phe-NH2), DAMGO (Tyr-D-Ala-Gly-N-MePhe-Gly-ol), Leu-enkephalin (Tyr-Gly-Gly-Phe-Leu), Met-enkephalin (Tyr-Gly-Gly-Phe-Met), Glycyl-L-proline (Gly-Pro), L-alanyl-L-proline (Ala-Pro), Cysteinylglycine (Cys-Gly), Glycyltyrosine (Gly-Tyr), Valyltyrosine (Val-Tyr), Isoleucyltyrosine (Ile-Tyr), Leucyltyrosine (Leu-Tyr), Methionylglycine (Met-Gly), Prolylhydroxyproline (Pro-Hyp), Glycylsarcosine (Gly-Sar), Alanylglutamine (Ala-Gln), Glycylhistidine (Gly-His), Histidylglycine (His-Gly), Tyrosylglycine (Tyr-Gly), Phenylalanylglycine (Phe-Gly), Tryptophylglycine (Trp-Gly), Lysylglycine (Lys-Gly), Arginylglycine (Arg-Gly), Aspartylglycine (Asp-Gly), Glutamylglycine (Glu-Gly), Serylglycine (Ser-Gly). Threonylglycine (Thr-Gly), Cysteinylglycine (Cys-Gly), Prolylglycine (Pro-Gly), Valylglycine (Val-Gly), Leucylglycine (Leu-Gly), Isoleucylglycine (Ile-Gly), Methionylglycine (Met-Gly), Glycylleucine (Gly-Leu), Glycylisoleucine (Gly-Ile), Glycylvaline (Gly-Val), Glycylmethionine (Gly-Met), Glycylphenylalanine (Gly-Phe), Glycyltryptophan (Gly-Trp), Glycyllysine (Gly-Lys), Glycylarginine (Gly-Arg), Glycylaspartic acid (Gly-Asp), Glycylglutamic acid (Gly-Glu), Glycylserine (Gly-Ser), Glycylthreonine (Gly-Thr), Glycylcysteine (Gly-Cys), Glycylproline (Gly-Pro), Alanylglycine (Ala-Gly), Valylglycine (Val-Gly), Leucylglycine (Leu-Gly), Isoleucylglycine (Ile-Gly), Prolylglycine (Pro-Gly), Phenylalanylglycine (Phe-Gly), Tryptophanylglycine (Trp-Gly), Tyrosylglycine (Tyr-Gly), Histidylglycine (His-Gly), Lysylglycine (Lys-Gly), Arginylglycine (Arg-Gly), Aspartylglycine (Asp-Gly), Glutamylglycine (Glu-Gly), Serylglycine (Ser-Gly), Threonylglycine (Thr-Gly), Cysteinylglycine (Cys-Gly), Methionylglycine (Met-Gly), Glycylhistidine (Gly-His), Histidylalanine (His-Ala), Histidylleucine (His-Leu), Histidylisoleucine (His-Ile), Histidylvaline (His-Val), Histidylmethionine (His-Met), Histidylphenylalanine (His-Phe), Histidyltryptophan (His-Trp), Histidyltyrosine (His-Tyr), Histidyllysine (His-Lys), Histidylarginine (His-Arg), Histidylaspartic acid (His-Asp), Histidylglutamic acid (His-Glu), Histidylserine (His-Ser), Histidylthreonine (His-Thr), Histidylcysteine (His-Cys), Histidylproline (His-Pro), Alanylhistidine (Ala-His), Valylhistidine (Val-His), Leucylhistidine (Leu-His), Isoleucylhistidine (Ile-His), His-Gly-Ala-Lys, Trp-His-Gly, His-His-Gly, His-Ala-His, Trp-His-Trp-His, Tyr-His, Gly-Pro-Glu, Lys-Pro-Val, Ile-Pro-Pro, Val-Pro-Pro, Arg-Gly-Asp, Gly-His-Lys, Lys-Gly-His, Pro-Leu-Gly, Thr-Lys-Pro, Glu-Cys-Gly, Leu-Gly-Pro, Ser-Lys-Pro, Ala-Gly-Ser, Gly-Gly-His, Lys-Val-Leu, Arg-Pro-Lys, Tyr-Gly-Gly, His-Ala-Lys, Pro-Hyp-Gly, Glu-Ala-Gln, Thr-Lys-Pro-Arg, Arg-Gly-Asp-Ser, Gly-Pro-Hyp-Gly, Tyr-Pro-Phe-Phe, Tyr-Pro-Trp-Phe, Ala-Gly-Ser-Glu, Lys-Phe-Lys-Phe, Gly-Gln-Pro-Arg, Arg-Glu-Asp-Val, Pro-Gly-Pro-Gly, Lys-Leu-Leu-Gly, Asp-Glu-Val-Asp, Gly-His-Lys-Cu, Ala-Lys-Glu-Phe, Val-Gly-Val-Ala, Arg-Pro-Lys-Pro, Gly-Pro-Arg-Pro, Leu-Ser-Gly-Ala, His-Phe-Arg-Trp, Asp-Phe-Asp-Tyr, and any salts thereof.

In some embodiments, the antioxidant comprises L-carnosine, D-carnosine, acetyl-carnosine, anserine, alanine, L-histidine, D-histidine, a derivative thereof, or a combination thereof. In some embodiments, the antioxidant comprises carnosine. In some embodiments, the antioxidant comprises anserine. In some embodiments, the antioxidant comprises L-carnosine. In some embodiments, the intranasal composition comprises, by weight of the total composition, 0.5%-40% (e.g., 0.5, 0.7, 1, 1.5, 2, 2.5, 3, 3.5, 4, 4.5, 5, 5.5, 6, 6.5, 7, 7.5, 8, 8.5, 9, 9.5, 10% or any value therebetween) antioxidant. In some embodiments, the intranasal composition comprises, by weight of the total composition, 0.5%-40% (e.g., 0.5, 0.7, 1, 1.5, 2, 2.5, 3, 3.5, 4, 4.5, 5, 5.5, 6, 6.5, 7, 7.5, 8, 8.5, 9, 9.5, 10% or any value therebetween) carnosine. In some embodiments, the intranasal composition comprises, by weight of the total composition, 0.5%-40% (e.g., 0.5, 0.7, 1, 1.5, 2, 2.5, 3, 3.5, 4, 4.5, 5, 5.5, 6, 6.5, 7, 7.5, 8, 8.5, 9, 9.5, 10% or any value therebetween) L-carnosine.

In some embodiments, the peptide is capable of forming an inclusion complex with one or more cyclodextrins. As used herein, an “inclusion complex” means a complex wherein the the inner cavity of the cyclodextrin is occupied (at least partially) by a guest molecule (e.g., such as a peptide).

### Surfactant

In some embodiments, the intranasal composition comprises a surfactant, such as a non-ionic surfactant. In some embodiments, intranasal composition comprises one or more of the surfactants Polysorbate 80, Polysorbate 20, peg 40 hydrogenated castor oil. Surfactants also include but are not limited to: Polysorbate 80 NF, polyoxyethylene 20 sorbitan monolaurate, polyoxyethylene (4) sorbitan monolaurate, polyoxyethylene 20 sorbitan monopalmitate, polyoxyethylene 20 sorbitan monostearate, polyoxyethylene (4) sorbitan monostearate, polyoxyethylene 20 sorbitan tristearate, polyoxyethylene (5) sorbitan monooleate, polyoxyethylene 20 sorbitan trioleate, polyoxyethylene 20 sorbitan monoisostearate, sorbitan monooleate, sorbitan monolaurate, sorbitan monopalmitate, sorbitan monostearate, sorbitan trilaurate, sorbitan trioleate, sorbitan tristearate, and the like, and mixtures thereof. Due to their chemical properties, certain surfactants can function as a preservatives and/or absorption enhancers in certain circumstances, depending on concentration in the formulation and other factors.

Surfactants include but are not limited to: cationic, anionic, nonionic and zwitterionic surfactants. In some embodiments, the surfactants are layered (e.g., anionic-non-ionic-cationic) to prevent incompatibility. In some embodiments, layered surfactants comprise micelle entrapment of a therapeutic agent.

Surfactants also include: anionic surfactants (e.g. carboxylates sulphonates, petroleum sulphonates, alkylbenzenesulphonates, naphthalenesulphonates, olefin sulphonates, alkyl sulphates, sulphates, sulphated natural oils and fats, sulphated esters, sulphated alkanolamides, alkylphenols, ethoxylated and sulphated), nonionic surfactants (e.g. ethoxylated aliphatic alcohol, polyoxyethylene surfactants, carboxylic esters, polyethylene glycol esters, anhydrosorbitol ester and it's ethoxylated derivatives, glycol esters of fatty acids, carboxylic amides, monoalkanolamine condensates, polyoxyethylene fatty acid amides), cationic surfactants (e.g. quaternary ammonium salts, amines with amide linkages, polyoxyethylene alkyl and alicyclic amines, 4.n,n,n′,n′ tetrakis substituted ethylenediamines, 2-alkyl 1-hydroxethyl 2-imidazolines), amphoteric surfactants (amphoteric surfactants contains both an acidic and a basic hydrophilic moiety in their surface e.g., n-coco 3-aminopropionic acid/sodium salt, n-tallow 3-iminodipropionate, disodium salt, n-carboxymethyl n dimethyl n-9 octadecenyl ammonium hydroxide, n-cocoamidethyl n hydroxyethylglycine, sodium salt, etc.).

In some embodiments, the intranasal composition comprises, by weight of the total composition 0.01%-1% (such as 0.01, 0.02, 0.03, 0.04, 0.05, 0.06, 0.07% or any value therebetween) surfactant. In some embodiments, the intranasal composition comprises, by weight of the total composition 0.01%-1% (such as 0.01, 0.02, 0.03, 0.04, 0.05, 0.06, 0.07% or any value therebetween) polysorbate 80. In some embodiments, the intranasal composition comprises, by weight of the total composition 0.01%-1% (such as 0.01, 0.02, 0.03, 0.04, 0.05, 0.06, 0.07% or any value therebetween) polysorbate 20. In some embodiments, the intranasal composition comprises, by weight of the total composition 0.01%-1% (such as 0.01, 0.02, 0.03, 0.04, 0.05, 0.06, 0.07% or any value therebetween) peg 40 hydrogenated castor oil.

### Preservative

In some embodiments, the intranasal composition comprises a preservative. As used herein the term “preservative” refers to any known pharmaceutically acceptable preservative that functions by inhibiting bacteria, fungi, yeast, mold, other microbe. Suitable preservatives include but are not limited to antimicrobial agents. In some embodiments, antimicrobial agents comprise potassium sorbate, sodium benzoate, paraben, benzyl alcohol, sorbic acid, triclosan, phenoxyisopropanol, diazolidinyl urea, bronopol, Alkyl (C12-22) trimethyl ammonium bromide, Alkyl (C12-22) trimethyl ammonium chloride, Benzalkonium chloride, Benzalkonium bromide, Benzalkonium saccharinate, ethylhexylglycerin, phenoxyethanol, or a combination thereof. Where the intranasal compositions disclosed herein comprise a preservative, the preservative may be selected from any pharmaceutically acceptable preservative. In still other embodiments, the intranasal compositions disclosed herein do not contain a preservative.

In some embodiments, the intranasal composition comprises, by weight of the total composition 0.05%-0.5% (such as 0.1, 0.12, 0.13, 0.14, 0.15% or any value therebetween) preservative. In some embodiments, the intranasal composition comprises, by weight of the total composition 0.05%-0.5% (such as 0.1, 0.12, 0.13, 0.14, 0.15% or any value therebetween) potassium sorbate.

In some embodiments, the intranasal composition comprises a chelator, such as EDTA or 2,3-dimercaptopropanesulfonic acid. In some embodiments, the intranasal composition comprises, by weight of the total composition, 0.001%-0.05% (such as 0.005, 0.01, 0.015, 0.2% or any value therebetween) chelator. In some embodiments, the intranasal composition comprises, by weight of the total composition 0.001%-0.05% (such as 0.005, 0.01, 0.015, 0.2% or any value therebetween) EDTA.

### Cyclic Oligosaccharide-Based Polymer

In some embodiments, the intranasal composition comprises a cyclic oligosaccharide-based polymer. In some embodiments, the cyclic oligosaccharide-based polymer comprises an alpha cyclodextrin, beta cyclodextrin, a gamma cyclodextrin, a 2-hydroxypropyl-β-cyclodextrin, a β-cyclodextrin sulfobutylether, hydroxyethyl-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-1-β-cyclodextrin, glycosyl-β-cyclodextrin, maltosyl-β-cyclodextrin or any derivative thereof, and any combination thereof. In some embodiments, the cyclic oligosaccharide-based polymer is non-crosslinked. In some embodiments, the cyclic oligosaccharide-based polymer is crosslinked. In some embodiments, the cyclic oligosaccharide-based polymer is an alkylated derivative. In some embodiments, the intranasal composition comprises, by weight of the total composition, 0.1%-20% (0.1, 0.5, 0.7, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20% or any value therebetween) cyclic oligosaccharide-based polymer. In some embodiments, the intranasal composition comprises, by weight of the total composition, 0.1%-20% (0.1, 0.5, 0.7, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20% or any value therebetween) cyclodextrin or an alkylated derivative thereof. In some embodiments, the intranasal composition comprises, by weight of the total composition, 0.1%-20% (0.1, 0.5, 0.7, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20% or any value therebetween) gamma cyclodextrin or an alkylated derivative thereof.

In some embodiments, the cyclic oligosaccharide-based polymer comprises gamma cyclodextrin. In some embodiments, the gamma cyclodextrin comprises non-crosslinked gamma cyclodextrin. In some embodiments, the gamma cyclodextrin comprises crosslinked gamma cyclodextrin.

In some embodiments, the alpha cyclodextrin has the following chemical formula:

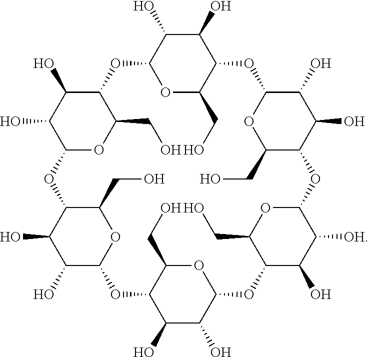 [SMILES: OC[C@H]1O[C@@H]2O[C@H]3[C@H](O)[C@@H](O)[C@@H](O[C@H]4[C@H](O)[C@@H](O)[C@@H](O[C@H]5[C@H](O)[C@@H](O)[C@@H](O[C@H]6[C@H](O)[C@@H](O)[C@@H](O[C@H]7[C@H](O)[C@@H](O)[C@@H](O[C@H]1[C@H](O)[C@H]2O)O[C@@H]7CO)O[C@@H]6CO)O[C@@H]5CO)O[C@@H]4CO)O[C@@H]3CO]

In some embodiments, the beta cyclodextrin has the following chemical formula:

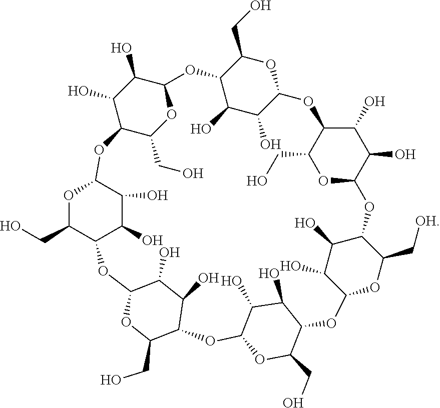 [SMILES: OC[C@H]1O[C@@H]2O[C@H]3[C@H](O)[C@@H](O)[C@@H](O[C@H]4[C@H](O)[C@@H](O)[C@@H](O[C@H]5[C@H](O)[C@@H](O)[C@@H](O[C@H]6[C@H](O)[C@@H](O)[C@@H](O[C@H]7[C@H](O)[C@@H](O)[C@@H](O[C@H]8[C@H](O)[C@@H](O)[C@@H](O[C@H]1[C@H](O)[C@H]2O)O[C@@H]8CO)O[C@@H]7CO)O[C@@H]6CO)O[C@@H]5CO)O[C@@H]4CO)O[C@@H]3CO]

In some embodiments, the gamma cyclodextrin has the following chemical formula:

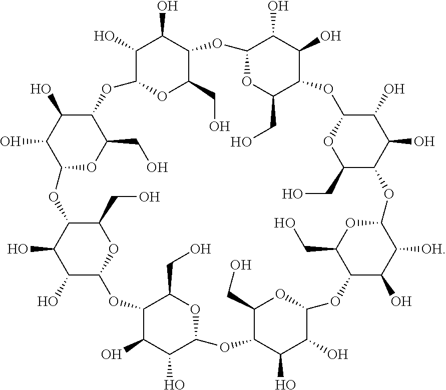 [SMILES: OC[C@H]1O[C@@H]2O[C@H]3[C@H](O)[C@@H](O)[C@@H](O[C@H]4[C@H](O)[C@@H](O)[C@@H](O[C@H]5[C@H](O)[C@@H](O)[C@@H](O[C@H]6[C@H](O)[C@@H](O)[C@@H](O[C@H]7[C@H](O)[C@@H](O)[C@@H](O[C@H]8[C@H](O)[C@@H](O)[C@@H](O[C@H]9[C@H](O)[C@@H](O)[C@@H](O[C@H]1[C@H](O)[C@H]2O)O[C@@H]9CO)O[C@@H]8CO)O[C@@H]7CO)O[C@@H]6CO)O[C@@H]5CO)O[C@@H]4CO)O[C@@H]3CO]

In some embodiments, the intranasal composition disclosed herein comprises a pH modifying agent. In some embodiments, the composition has a pH between 7.5 and 9.0 (e.g., pH 8.0, 8.1, 8.2, 8.3, 8.4, 8.5 or any value therebetween). In some embodiments, the composition has a pH between 8.25 and 8.31 (e.g., pH 8.25, 8.26, 8.27, 8.28, 8.29, 8.3, 8.31, or any value therebetween). In some embodiments, the intranasal composition disclosed herein lacks a pH modifying agent and has a pH between 7.5 and 9.0 (e.g., pH 8.0, 8.1, 8.2, 8.3, 8.4, 8.5 or any value therebetween).

### Particle Characteristics

In some embodiments, the intranasal composition comprises particles. In some embodiments, the particles may be characterized by mean diameter (or mean particle size) and/or particle size distribution of a population of particles. For example, measures that may be derived from the particle size distribution include the mean and modal diameter. The d90 and d10 are the diameters below which 90% and 10% of the particles fall. The value d50 is the median diameter, or diameter below which 50% of the particles fall, and d75 and d25 are the diameters below which 75% and 25% of the particles fall, respectively. The size and distribution of a particle population can be measured using laser light scattering methods. Instruments suitable for measuring the size and distribution of a particle population are commercially available, from, for example, Malvern Instruments Ltd. Particle containing a therapeutic agent, such as those disclosed herein, may be spherical or substantially spherical. In these instances, the particle size(s) measured and reported by such instruments for a sample of particles will essentially represent the diameter(s) of the particles. The size and distribution of a particle population can also be measured manually using light microscopy and size metrics (e.g. micron ruler).

In some embodiments, the span (d90-d10) of the particles disclosed herein may be no more than about 90 μm. In some embodiments, the span (d90-d10) of the particles disclosed herein may be no more than about 70 μm. In some embodiments, the span (d90-d10) of the particles disclosed herein may be no more than about 50 μm.

In some embodiment, the particles disclosed herein have a unimodal particle size distribution and the diameters of particles deviate from the modal diameter by no more than about 90 μm. In some embodiment, the particles disclosed herein have a bimodal particle size distribution and the diameters of particles deviate from the modal diameter by no more than about 90 μm.

In some embodiments, the particles disclosed herein have a mean diameter of less than 100 μm. In some embodiments, the particles disclosed herein have a mean diameter of less than 70 μm. In some embodiments, the particles disclosed herein have a mean diameter of less than 50 μm. In some embodiments, the particles disclosed herein have a mean diameter of less than 30 μm. In some embodiments, the particles disclosed herein have a mean diameter of less than 20 μm. In some embodiments, the particles disclosed herein have a mean diameter of less than 10 μm. In some embodiments, the particles disclosed herein have a mean diameter of about 3 μm to 20 μm.

In some embodiments, 90% of the particles are 20 μm or less in diameter. In some embodiments, 90% of the particles are 15 μm or less in diameter. In some embodiments, 90% of the particles are 10 μm or less in diameter. In some embodiments, 90% of the particles are 5 μm or less in diameter.

In some embodiments, intranasal composition disclosed herein comprises a humectant such as one or more of an amino acid, aloe vera extract, a fatty acid, hyaluronic acid (HA), collagen, silicone, a disaccharide (e.g., sucrose or trehalose), maltitol, erythrol, sorbitol, glycerin, propanediol, propylene glycol, glycerin or any other glycol/diol. In some embodiments, the humectant comprises glycerin and/or HA. In some embodiments, HA is swapped for any other heavy molecular weight polymer such as collagen or a derivative thereof. In some embodiments, collagen is interchangeable with HA or can be combined with HA, e.g., 0.6% HA combined with 0.5% collagen. In some embodiments, the composition comprises 0.1-5% of HA, 0.1-5% of collagen or derivatives thereof, or 0.1-5% of HA and collagen or a derivative thereof combined. In some embodiments, the intranasal composition comprises 0.1-1% (e.g., 0.1, 0.2, 0.3, 0.4, 0.5, 0.6, 0.7% or any number therebetween) of hyaluronic acid.

In some embodiments, the particles disclosed herein are surfactant-based particles. These particles may vary in size, shape, composition, and properties depending on the specific surfactants used, their concentration, and the preparation methods employed. In some cases, combinations of different surfactants or additional components may be used to create more complex or multifunctional particles. In some embodiments, the surfactant-based particles comprise one or more of micelles, reverse micelles, vesicles (liposomes), niosomes, nanoemulsions, microemulsions, pickering emulsions, multiple emulsions (e.g. water-in-oil-in-water), and solid lipid nanoparticles.

### Example Formulations

In some embodiments, the composition disclosed herein comprises:

3%-25% carnosine
3%-10% cyclodextrin
0.015%-0.09% polysorbate 80
0.001%-0.05% EDTA
0.012%-0.24% potassium sorbate
0.1%-4% Carboxymethyl cellulose (CMC)

In some embodiments, the composition disclosed herein comprises one or more of:

3%-25% carnosine
3%-10% cyclodextrin
0.015%-0.09% polysorbate 80
0.001%-0.05% EDTA
0.012%-0.24% potassium sorbate
0.1%-4% Carboxymethyl cellulose (CMC)
0.1%-5% glycerin
0.001%-0.05% polysorbate 20
0.001%-0.05% peg 40 hydrogenated castor oil
0.05%-3% hyaluronic acid
0.05%-3% sodium poly-gamma-glutamate

In some embodiments, the composition disclosed herein comprises:

15% Carnosine
5% Gamma-cyclodextrin or alkylated derivative hydroxylpropyl
0.05% Polysorbate 80
0.01% EDTA
0.12% Potassium sorbate
0.8% Carboxymethyl cellulose (CMC)

In some embodiments, the composition disclosed herein comprises:

20% Carnosine
5% Gamma-cyclodextrin or alkylated derivative hydroxylpropyl
1% Glycerin
0.05% Polysorbate 80
0.01% polysorbate 20
0.01% EDTA
0.12% Potassium sorbate
1% Carboxymethyl cellulose (CMC)

In some embodiments, the composition disclosed herein comprises:

15% Carnosine
5% hydroxylpropyl beta-cyclodextrin
0.05% Polysorbate 80
0.01% peg 40 hydrogenated castor oil
0.01% EDTA
0.12% Potassium sorbate
0.5% glycerin
0.5% Carboxymethyl cellulose (CMC)
0.5% hyaluronic acid

In some embodiments, the composition disclosed herein comprises:

15% Carnosine
5% hydroxylpropyl beta-cyclodextrin
0.05% Polysorbate 80
0.01% peg 40 hydrogenated castor oil
0.01% EDTA
0.12% Potassium sorbate
0.5% glycerin
0.5% Carboxymethyl cellulose (CMC)
0.5% sodium poly-gamma-glutamate

In some embodiments, the composition disclosed herein comprises:

5% Carnosine
1% Carboxymethyl cellulose (CMC)
0.025% Polysorbate 80
0.12% Potassium sorbate
0.01% EDTA

In some embodiments, the composition disclosed herein comprises:

5% Carnosine
1.5% Carboxymethyl cellulose (CMC)
0.025% Polysorbate 80
0.12% Potassium sorbate
0.01% EDTA

In some embodiments, the composition disclosed herein comprises:

5% Carnosine
2% Carboxymethyl cellulose (CMC)
0.025% Polysorbate 80
0.12% Potassium sorbate
0.01% EDTA

In some embodiments, the composition disclosed herein comprises:

5% Carnosine
1% Carboxymethyl cellulose (CMC)
0.025% Polysorbate 80
0.12% Potassium sorbate
0.01% EDTA
5% Cyclodextrin (e.g. Cavamax ® W8)

In some embodiments, the composition disclosed herein comprises:

5% Carnosine
2% Carboxymethyl cellulose (CMC)
0.025% Polysorbate 80
0.12% Potassium sorbate
0.01% EDTA
5% Cyclodextrin (e.g. Cavamax ® W8)

In some embodiments, the composition disclosed herein comprises:

10% Carnosine
1% Carboxymethyl cellulose (CMC)
0.025% Polysorbate 80
0.12% Potassium sorbate
0.01% EDTA
5% Cyclodextrin (e.g. Cavitron ™ W7 HP5)

In some embodiments, the composition disclosed herein comprises:

10% Carnosine
2% Carboxymethyl cellulose (CMC)
0.025% Polysorbate 80
0.12% Potassium sorbate
0.01% EDTA
5% Cyclodextrin (e.g. Cavitron ™ W7 HP5)

According to some aspects, the present disclosure provides an intranasal composition made by the process of: dissolving carnosine in water to obtain a clear solution; adding cyclodextrin under stirring to form a complex with carnosine; adding a surfactant (e.g. P80) to form a hybrid micelle and cyclodextrin complex; and adding CMC with mixing to maintain the viscosity of formulation. In some embodiments, potassium sorbate is then added. In some embodiments, EDTA is added.

According to some aspects, the present disclosure provides an intranasal composition comprising one or more of:

| Ingredients | % w/w (g) |
|---|---|
| Carnosine (S212) | 5% |
| Carboxymethyl cellulose (CMC) | 1%, 1.5%, 2% |
| P80 | 0.025% |
| Potassium sorbate | 0.12% |
| EDTA | 0.01% |

made by the process of: dissolving carnosine in water to obtain a clear solution; adding P80 to form a carnosine micelle preparation; adding CMC and mixing well to maintain the viscosity of formulation. In some embodiments, potassium sorbate is then added and/or EDTA is added.

According to some aspects, the present disclosure provides an intranasal composition comprising one or more of:

| Ingredients | % w/w (g) |
|---|---|
| Carnosine S212 | 5% |
| CMC | 1%, 2% |
| P80 | 0.025% |
| Potassium sorbate | 0.12% |
| EDTA | 0.01% |
| CD (Cavamax ® W8) | 5% |

made by the process of: dissolving carnosine in water to obtain a clear solution; adding cyclodextrin under stirring to form a complex with carnosine; adding P80 to form a hybrid micelle and cyclodextrin complex; and adding CMC with mixing to maintain the viscosity of formulation. In some embodiments, potassium sorbate is then added. In some embodiments, EDTA is added.

According to some aspects, the present disclosure provides an intranasal composition comprising one or more of:

| Ingredients | % w/w (g) |
|---|---|
| Carnosine S212 | 10% |
| CMC | 1%, 2% |
| P80 | 0.025% |
| Potassium sorbate | 0.12% |
| EDTA | 0.01% |
| CD (Cavitron ™ W7 HP5) | 5% |

made by the process of: dissolving carnosine in water to obtain a clear solution; adding cyclodextrin under stirring to form a complex with carnosine; adding P80 to form a hybrid micelle and cyclodextrin complex; and adding CMC with mixing to maintain the viscosity of formulation. In some embodiments, potassium sorbate is then added. In some embodiments, EDTA is added.

In some embodiments, the intranasal compositions disclosed herein comprises sweetners/flavoring agents/task-masking agents which include, but are not limited to, sucrose, dextrose, lactose, sucralose, acesulfame-K, aspartame, saccharin, sodium saccharin, citric acid, aspartic acid, eucalyptol, mannitol, glycerin, xylitol, menthol, glycyrrhizic acid, cinnamon oils, oil of wintergreen, peppermint oils, clover oil, bay oil, anise oil, eucalyptus, vanilla, citrus oil such as lemon oil, orange oil, grape and grapefruit oil, fruit essences including apple, peach, pear, strawberry, raspberry, cherry, plum, pineapple, apricot, etc. and combinations thereof. In some embodiments, the formulations contain from about 0.0001 percent to about 1 percent of a sweetener/flavoring agent/task-masking agent, and may be present at lower or higher amounts as a factor of one or more of potency of the effect on flavor, solubility of the flavorant, effects of the flavorant on solubility or other physicochemical or pharmacokinetic properties of other formulation components, or other factors.

In some embodiments, the intranasal compositions disclosed herein comprise an antioxidant, surfactant, co-solvent, adhesive, stabilizer, osmolarity adjusting agent, preservative, penetration enhancer, chelating agent, sweetening agent, flavoring agent, taste masking agent, or colorant. Furthermore, some agents or components of the intranasal compositions disclosed herein may concurrently act, for example, as both a pH modifying agent and an osmolarity adjusting agent or as both sensory agent and a co-solvent. Where a given agent or component of an intranasal formulation is described herein with respect to a particular function, it is in no way taken to be limited to a single function only. It would be understood by a person skilled in the art that agents or components may additionally perform alternative or multiple functions.

In some embodiments, the intranasal compositions disclosed herein may be administered to a subject in need thereof, together with other medication for a discrete period of time, to address specific symptoms. In still other embodiments, the person in need thereof may be treated with both an intranasal composition disclosed herein and one or more additional medications (administered sequentially or in combination) for the duration of the treatment period. Such combination therapy may be particularly useful, for example, where an additive or synergistic therapeutic effect is desired.

### Lyophilization

In some embodiments, the formulations disclosed herein are lyophilized. In some embodiments, lyophilized formulations comprise compositions that can protect the integrity and/or structure of the formulation (i.e. a lyoprotectant). In some embodiments, the lyoprotectant is as disclosed in U.S. Pat. No. 11,833,224, which is incorporated by reference in its entirety. In some embodiments, formulations are lyophilized using the compositions of the instant disclosure to retain at least 90%, 91%, 92%, 93%, 94%, 95%, 96%, 97%, 98%, 99% or more of their biological activity and/or integrity when reconstituted/thawed. In a specific embodiment, the compositions of the instant disclosure are effective at protecting biological activity and/or structural integrity of surfactant-based particles at least 90%, 91%, 92%, 93%, 94%, 95%, 96%, 97%, 98%, 99% or more as compared to surfactant-based particles that have not been lyophilized.

In some embodiments, the lyoprotectant composition comprises 0.3%-8% (e.g., 0.3, 0.5, 0.7, 0.9, 1, 1.2, 1.5, 1.7, 2, 2.2, 2.5, 2.7, 3, 3.2, 3.5, 3.7, 4, 4.2, 4.5, 4.7, 5, 5.2, 5.5, 5.7, 6, 6.2, 6.5, 6.7, 7, 7.2, 7.5, 7.7, 8%, or any value therebetween) cyclic oligosaccharide-based polymer, 2%-10% (e.g., 2, 2.2, 2.5, 2.7, 3, 3.2, 3.5, 3.7, 4, 4.2, 4.5, 4.7, 5, 5.2, 5.5, 5.7, 6, 6.2, 6.5, 6.7, 7, 7.2, 7.5, 7.7, or 8, 8.2, 8.5, 8.7, 9, 9.2, 9.5, 9.7, 9.9, 10%, or any value therebetween) sugar, and 0.2%-10% (e.g., 0.2, 0.3, 0.5, 0.7, 0.9, 1, 1.2, 1.5, 1.7, 2, 2.2, 2.5, 2.7, 3, 3.2, 3.5, 3.7, 4, 4.2, 4.5, 4.7, 5, 5.2, 5.5, 5.7, 6, 6.2, 6.5, 6.7, 7, 7.2, 7.5, 7.7, 8, 8.2, 8.5, 8.7, 9, 9.2, 9.5, 9.7, 9.9, 10%, or any value therebetween) amino acid.

In some embodiments, the lyoprotectant composition comprises 1%-5% (e.g., 1, 1.2, 1.5, 1.7, 2, 2.2, 2.5, 2.7, 3, 3.2, 3.5, 3.7, 4, 4.2, 4.5, 4.7, 5, or any value therebetween) cyclic oligosaccharide-based polymer. In some embodiments, the lyoprotectant composition comprises 1%-3% (e.g., 1, 1.2, 1.5, 1.7, 2, 2.2, 2.5, 2.7, 3, or any value therebetween) cyclic oligosaccharide-based polymer. In some embodiments, the composition comprises 2% cyclic oligosaccharide-based polymer.

In some embodiments, the lyoprotectant composition comprises 4%-8% (e.g., 4, 4.2, 4.5, 4.7, 5, 5.2, 5.5, 5.7, 6, 6.2, 6.5, 6.7, 7, 7.2, 7.5, 7.7, 8%, or any value therebetween) sugar. In some embodiments, the lyoprotectant composition comprises 5%-7% (e.g., 5, 5.2, 5.5, 5.7, 6, 6.2, 6.5, 6.7, 7, or any value therebetween) sugar. In some embodiments, the composition comprises 6% sugar.

In some embodiments, the lyoprotectant composition comprises 0.2%-4% (e.g., 0.2, 0.3, 0.5, 0.7, 0.9, 1, 1.2, 1.5, 1.7, 2, 2.2, 2.5, 2.7, 3, 3.2, 3.5, 3.7, 4, or any value therebetween) amino acid. In some embodiments, the lyoprotectant composition comprises 0.3%-0.5% (e.g., 0.3, 0.4 or 0.5%) amino acid. In some embodiments, the lyoprotectant composition comprises 0.5% amino acid.

In a specific embodiment, the lyoprotectant composition comprises about 6% sucrose, about 2% gamma cyclodextrin and about 0.5% trimethylglycine. In a specific embodiment, the lyoprotectant composition comprises about 6% sucrose, about 1% gamma cyclodextrin and about 1% trimethylglycine. In some embodiments, the lyoprotectant composition ingredients are constituted in water or phosphate buffered saline (PBS). In some embodiments, the lyoprotectant is sterilized, optionally by filtering through a 0.2 micron filter.

In some embodiments the compositions disclosed herein comprise 0.3%-8% (e.g., 0.3, 0.5, 0.7, 0.9, 1, 1.2, 1.5, 1.7, 2, 2.2, 2.5, 2.7, 3, 3.2, 3.5, 3.7, 4, 4.2, 4.5, 4.7, 5, 5.2, 5.5, 5.7, 6, 6.2, 6.5, 6.7, 7, 7.2, 7.5, 7.7, 8%, or any value therebetween) cyclic oligosaccharide-based polymer, 2%-10% (e.g., 2, 2.2, 2.5, 2.7, 3, 3.2, 3.5, 3.7, 4, 4.2, 4.5, 4.7, 5, 5.2, 5.5, 5.7, 6, 6.2, 6.5, 6.7, 7, 7.2, 7.5, 7.7, 8, 8.2, 8.5, 8.7, 9, 9.2, 9.5, 9.7, 9.9, 10%, or any value therebetween) sugar, 0.2%-10% (e.g., 0.2, 0.3, 0.5, 0.7, 0.9, 1, 1.2, 1.5, 1.7, 25 2, 2.2, 2.5, 2.7, 3, 3.2, 3.5, 3.7, 4, 4.2, 4.5, 4.7, 5, 5.2, 5.5, 5.7, 6, 6.2, 6.5, 6.7, 7, 7.2, 7.5, 7.7, 8, 8.2, 8.5, 8.7, 9, 9.2, 9.5, 9.7, 9.9, 10%, or any value therebetween) amino acid, and a surfactant-based particle.

In some embodiments, the composition comprises 1%-5% (e.g., 1, 1.2, 1.5, 1.7, 2, 2.2, 2.5, 2.7, 3, 3.2, 3.5, 3.7, 4, 4.2, 4.5, 4.7, 5, or any value therebetween) cyclic oligosaccharide-based polymer. In some embodiments, the composition comprises 1%-3% (e.g., 1, 1.2, 1.5, 1.7, 2, 2.2, 2.5, 2.7, 3, or any value therebetween) cyclic oligosaccharide-based polymer. In some embodiments, the composition comprises 2% cyclic oligosaccharide-based polymer.

In some embodiments, the composition comprises 4%-8% (e.g., 4, 4.2, 4.5, 4.7, 5, 5.2, 5.5, 5.7, 6, 6.2, 6.5, 6.7, 7, 7.2, 7.5, 7.7, 8%, or any value therebetween) sugar. In some embodiments, the composition comprises 5%-7% (e.g., 5, 5.2, 5.5, 5.7, 6, 6.2, 6.5, 6.7, 7, or any value therebetween) sugar. In some embodiments, the composition comprises 6% sugar.

In some embodiments, the composition comprises 0.2%-4% (e.g., 0.2, 0.3, 0.5, 0.7, 0.9, 1, 1.2, 1.5, 1.7, 2, 2.2, 2.5, 2.7, 3, 3.2, 3.5, 3.7, 4, or any value therebetween) amino acid. In some embodiments, the composition comprises 0.3%-0.5% (e.g., 0.3, 0.4 or 0.5%) amino acid. In some embodiments, the composition comprises 0.5% amino acid. In a specific embodiment, the composition comprises about 6% sucrose, about 2% gamma cyclodextrin and about 0.5% trimethylglycine.

In some embodiments, the cyclic oligosaccharide-based polymer comprises an alpha cyclodextrin, beta cyclodextrin, a gamma cyclodextrin, a 2-hydroxypropyl-β-cyclodextrin, a β-cyclodextrin sulfobutylether, hydroxyethyl-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-1ß-cyclodextrin, glocosyl-β-cyclodextrin, maltosyl-β-cyclodextrin or any derivative thereof, and any combination thereof. In some embodiments, the cyclic oligosaccharide-based polymer comprises gamma cyclodextrin. In some embodiments, the alpha cyclodextrin has the chemical formula as disclosed herein. In some embodiments, the beta cyclodextrin has the chemical formula as disclosed herein. In some embodiments, the gamma cyclodextrin has the chemical formula as disclosed herein. In some embodiments, the sugar comprises sucrose, mannitol, and/or trehalose. In some embodiments, the sugar comprises sucrose. In some embodiments, the amino acid comprises trimethylglycine, glycine, arginine or any salts thereof.

In some embodiments, the surfactant-based particle (e.g. liposome or noisome) stabilized using the lyoprotectant composition of the instant disclosure during lyophilization retains at least 90% (e.g., at least 90%, 91%, 92%, 93%, 94%, 95%, 96%, 97%, 98%, 99% or more) of its structural integrity when resolubilized. In some embodiments, the lyoprotectant composition reduces agglomeration and aggregation of the biologic drug during lyophilization by at least 90% (e.g., at least 90%, 91%, 92%, 93%, 94%, 95%, 96%, 97%, 98%, 99% or more). In some embodiments, the biologic drug (exosome, liposome or noisome) stabilized using the lyoprotectant composition during lyophilization retains the bioavailability by at least 90% (e.g., at least 90%, 91%, 92%, 93%, 94%, 95%, 96%, 97%, 98%, 99% or more) when resolubilized.

In some embodiments, the surfactant-based particle is stabilized using the lyoprotectant composition of the instant disclosure during lyophilization, and the lyophilized composition comprising the surfactant-based particle when resolubilized has a zeta potential that is lower than-25 mV, or lower than-26 mV, or lower than-27 mV, or lower than-28 mV, or lower than-29 mV, or lower than-30 mV, or lower than-31 mV, or lower than-32 mV, or lower than-33 my, or lower than-34 mV, or lower than-35 mV, or lower than-36 mV, or lower than-37 mV, or lower than-38 mV, or lower than-39 mV, or lower than-40 mV. In some embodiments, a liposome or niosome composition is stabilized using the lyoprotectant composition of the instant disclosure during lyophilization, and the lyophilized composition comprising the liposome or niosome when resolubilized has a zeta potential that is lower than-25 mV, or lower than-26 mV, or lower than-27 mV, or lower than-28 mV, or lower than-29 mV, or lower than-30 mV, or lower than-31 mV, or lower than-32 mV, or lower than-33 mV, or lower than-34 mV, or lower than-35 mV, or lower than-36 mV, or lower than-37 my, or lower than-38 my, or lower than-39 mV, or lower than-40 mV.

In some embodiments, the surfactant-based particle is stabilized using the lyoprotectant composition of the instant disclosure during lyophilization, and the lyophilized composition comprising the surfactant-based particle when resolubilized has a zeta potential that is no more than 10 mV, no more than 9 mV, no more than 8 mV, no more than 7 mV, no more than 6 mV, no more than 5 mV, no more than 4 mV, no more than 3 mV, no more than 2 mV, or no more than 1 mV, higher than the zeta potential of a corresponding non-lyophilized composition comprising the surfactant-based particle. In some embodiments, a liposome or niosome composition is stabilized using the lyoprotectant composition of the instant disclosure during lyophilization, and the lyophilized composition comprising the liposome or niosome when resolubilized has a zeta potential that is no more than 10 mV, no more than 9 mV, no more than 8 mV, no more than 7 mV, no more than 6 mV, no more than 5 mV, no more than 4 mV, no more than 3 mV, no more than 2 mV, or no more than 1 mV, higher than the zeta potential of a corresponding non-lyophilized composition comprising the liposome or noisome before lyophilization.

According to some embodiments, the present disclosure provides a method for stabilizing a surfactant-based particle during lyophilization comprising mixing the surfactant-based particle with a lyoprotectant blend disclosed herein.

In some embodiments, the lyoprotectant is used to protect surfactant-based particles during lyophilization prior to addition of thickening agent (such as CMC). In some embodiments, the lyoprotectant is used to protect surfactant based particle during lyophilization after addition of thickening agent (such as CMC).

### Nasal Spray Delivery Devices

The formulations in accordance with the invention may be administered to a person in need thereof by any suitable intranasal delivery methods. Suitable methods for intranasal administration would be well-known to a person skilled in the art. The intranasal compositions disclosed herein can be administered as a spray or drop.

In some embodiments, suitable commercial packages containing the intranasal composition can be in any spray container known in the art. In one or more embodiments, the formulations in accordance with the invention may be administered via a spray device or container. Spray devices in accordance with the invention may be single unit dose systems or multiple dose systems, for example comprising a bottle, a pump and/or an actuator. Such spray devices are available commercially. Suitable commercial spray devices include those available from Nemera, Aptar, Bespak and Becton-Dickinson. In still other embodiments, the formulations in accordance with the invention may be administered via an electrostatic spray device. Other suitable means for administering formulations intranasally in accordance with the invention include via a dropper, a syringe, a squeeze bottle, and any other means known in the art for applying liquids to the nasal mucosa in an accurate and repeatable fashion.

The spray devices used to administer the composition can range from single-use metered-dose spray devices, multiple-use metered dose nasal spray devices and are not limited to spraying the solutions into each naris but can be administered as a gentle liquid stream from a plunger, syringe or the like or as drops from a unit-dose or multi-dose squeeze bottle, or other means known in the art for applying liquids to the nasal mucosa in an accurate and repeatable fashion.

According to some aspects, the present disclosure provides a nasal drug delivery device comprising a composition described herein. In some embodiments, the device is pre-primed. In some embodiments, the device can be primed before use. In some embodiments, the device can be actuated with one hand.

Nasal delivery is considered an attractive, safe, and easy-to-administer route for drug delivery. In addition, nasal delivery may help address issues related to poor bioavailability, slow absorption, drug degradation, and adverse events (AEs) in the gastrointestinal tract and avoids the first-pass metabolism in the liver.

Liquid nasal formulations are mainly aqueous solutions, but suspensions, emulsions, liposomes, micelles, and microspheres can also be delivered. Other liquid formulations can comprise liposomes, microspheres, mixed aqueous-organic formulations, non-aqueous formulations, dry powder and retentive formulations (gels). In traditional spray pump systems, antimicrobial preservatives are typically required to maintain microbiological stability in liquid formulations. In some embodiments, the nasal drug delivery device comprises metered spray pumps, which may deliver appropriate doses (e.g. 25-250 μL) per spray and provide high reproducibility of the emitted dose and plume geometry.

In traditional multi-use spray pump systems, antimicrobial preservatives are typically required to maintain microbiological stability in liquid formulations. However, preservative-free systems are also available, e.g. pumps comprising a filter membrane for air flow which prevents contamination. Additional nasal spray devices are optimized with dispenser tips that prevent clogging (useful for high-viscosity and high-volatile formulations), actuators that do not need re-priming after long periods of disuse, etc. Additional nasal spray devices are propellant driven. Yet additional nasal spray devices include dry powder inhalers.

The particle size and plume geometry can vary within certain limits and depend on the properties of the pump, the formulation, the orifice of the actuator, and the force applied. The droplet size distribution of a nasal spray is a critical parameter, since it significantly influences the in vivo deposition of the drug in the nasal cavity. The droplet size is influenced by the actuation parameters of the device and the formulation. The prevalent median droplet size should be between about 30 and about 100 μm. If the droplets are too large (>about 120 μm), deposition takes place mainly in the anterior parts of the nose. In its capacity as a surfactant, benzalkonium chloride and alkylmaltosides (e.g., a tetradecyl maltoside (TDM), a dodecyl maltoside (DDM), etc.) can affect the surface tension of droplets from a delivered nasal spray plume, producing spherical or substantially spherical particles having a narrow droplet size distribution (DSD), as well as the viscosity of a liquid formulation.

Plume geometry, droplet size and DSD of the delivered plume subsequent to spraying may be measured under specified experimental and instrumental conditions by appropriate and validated and/or calibrated analytical procedures known in the art. These include photography, laser diffraction, and impaction systems (cascade impaction, NGI). Plume geometry, droplet size and DSD can affect pharmacokinetic outcomes such as Cmax, Tmax, and dose proportionality.

Droplet size distribution can be controlled in terms of ranges for the D10, D50, D90, span [(D90-D10)/D50], and percentage of droplets less than 10 mm. In certain embodiments, the formulation will have a narrow DSD. In certain embodiments, the formulation will have a D (v, 50) of 30-70 μm and a D (v, 90)<100 μm.

In certain embodiments, the percent of droplets less than 10 μm will be less than 10%. In certain embodiments, the percent of droplets less than 10 μm will be less than 5%. In certain embodiments, the percent of droplets less than 10 μm will be less than 2%. In certain embodiments, the percent of droplets less than 10 μm will be less than 1%.

In certain embodiments, the formulation when dispensed by actuation from the device will produce a uniform circular plume with an ovality ratio close to 1. Ovality ratio is calculated as the quotient of the maximum diameter (Dmax) and the minimum diameter (Dmin) of a spray pattern taken orthogonal to the direction of spray flow (e.g., from the “top”). In certain embodiments, the ovality ratio is less than +2.0. In certain embodiments, the ovality ratio is less than +1.5. In certain embodiments, the ovality ratio is less than +1.3. In certain embodiments, the ovality ratio is less than +1.2. In certain embodiments, the ovality ratio is less than +1.1.

The details and mechanical principles of particle generation for different types of nasal aerosol devices has been described. See, Vidgren and Kublik, Adv. Drug Deliv. Rev. 29:157-77, 1998. Traditional spray pumps replace the emitted liquid with air, and preservatives are therefore required to prevent contamination. However, driven by the studies suggesting possible negative effects of preservatives, pump manufacturers have developed different spray systems that avoid the need for preservatives. These systems use a collapsible bag, a movable piston, or a compressed gas to compensate for the emitted liquid volume (on the World Wide Web at aptar.com and on the World Wide Web at rexam.com). The solutions with a collapsible bag and a movable piston compensating for the emitted liquid volume offer the additional advantage that they can be emitted upside down, without the risk of sucking air into the dip tube and compromising the subsequent spray. This may be useful for some products where the patients are bedridden and where a head-down application is recommended. Another method used for avoiding preservatives is that the air that replaces the emitted liquid is filtered through an aseptic air filter. In addition, some systems have a ball valve at the tip to prevent contamination of the liquid inside the applicator tip. More recently, pumps have been designed with side-actuation. The pump was designed with a shorter tip to avoid contact with the sensitive mucosal surfaces. New designs to reduce the need for priming and re-priming, and pumps incorporating pressure point features to improve the dose reproducibility and dose counters and lock-out mechanisms for enhanced dose control and safety are available (on the World Wide Web at rexam.com and on the World Wide Web at aptar.com).

Traditional, simple single, bi-dose and multi-use metered-dose spray pumps require priming and some degree of overfill to maintain dose conformity for the labeled number of doses. They are well suited for drugs to be administered daily over a prolonged duration, but due to the priming procedure and limited control of dosing, unless a specialty device is selected, they are less suited for drugs with a narrow therapeutic window of time in which to use the device, particularly if they are not used often. For expensive drugs and drugs intended for single administration or sporadic use and where tight control of the dose and formulation is of importance, single-dose (UDS) or bi-dose spray (BDS) devices are preferred (on the World Wide Web at aptar.com). A simple variant of a single-dose spray device (MAD™) is offered by LMA (LMA, Salt Lake City, Utah, USA; on the World Wide Web at Imana.com). A nosepiece with a spray tip is fitted to a standard syringe. The liquid drug to be delivered is first drawn into the syringe and then the spray tip is fitted onto the syringe. This device has been used in academic studies to deliver, for example, a topical steroid in patients with chronic rhinosinusitis and in a vaccine study. A pre-filled device based on the same principle for one or two doses (Accuspray™, Becton Dickinson Technologies, Research Triangle Park, N.C., USA; on the World Wide Web at bdpharma.com) is used to deliver the influenza vaccine FluMist™ (on the World Wide Web at flumist.com), approved for both adults and children in the US market. A similar device for two doses was marketed by a Swiss company for delivery of another influenza vaccine a decade ago.

Pre-primed single- and bi-dose devices are also available, and consist of a reservoir, a piston, and a swirl chamber (see, e.g., the UDS UnitDose™ and BDS BiDose™ devices from Aptar, formerly Pfeiffer). The spray is formed when the liquid is forced out through the swirl chamber. These devices are held between the second and the third fingers with the thumb on the actuator. A pressure point mechanism incorporated in some devices secures reproducibility of the actuation force and emitted plume characteristics. Currently, marketed nasal migraine drugs like Imitrex® (on the World Wide Web at gsk.com) and Zomig® (on the World Wide Web at az.com; Pfeiffer/Aptar single-dose device), the marketed influenza vaccine Flu-Mist (on the World Wide Web at flumist.com; Becton Dickinson single-dose spray device), and the intranasal formulation of naloxone for opioid overdose rescue, Narcan Nasal® (on the World Wide Web at narcan.com; Adapt Pharma) are delivered with this type of device.

In certain embodiments, the 90% confidence interval for dose delivered per actuation is ±about 2%. In certain embodiments, the 95% confidence interval for dose delivered per actuation is ±about 2.5%.

### Methods of Treatment

Several neurodegenerative diseases may be treated or prevented, or the effects minimized, using different embodiments of the intranasal compositions disclosed herein including, for example, Alzheimer's disease and Parkinson's disease. Generally, the treatment may be given in a single dose or multiple administrations, i.e., once, twice, three or more times daily over a period of time. For chronic disorders such as those diagnosed with, or at risk for, Alzheimer's disease, stroke or Parkinson's disease, the treatment may consist of at least one dose per day over an extended period of time.

The method disclosed herein delivers the therapeutic agent to the nasal cavity of a mammal. In some embodiments, the intranasal composition is delivered to the olfactory area in the upper one-third of the nasal cavity and, particularly, to the olfactory neuroepithelium in order to promote rapid and efficient delivery of the agent to the CNS along the olfactory neural pathway rather than the capillaries within the respiratory epithelium. This method allows direct delivery of active ingredients, such as carnosine, to the CNS.

To deliver the intranasal composition to the CNS, the intranasal composition alone or in combination with other substances as a pharmaceutical composition may be administered to the olfactory area located in the upper one-third of the nasal cavity. The intranasal composition may be administered intranasally as a liquid spray, nose drops, a gel or ointment, through a tube or catheter, by syringe, or by submucosal infusion. Optimization of the administration of the therapeutic agent is provided by the various embodiments by applying the agent to the upper third of the nasal cavity.

The optimal concentration of the active therapeutic agent will necessarily depend upon the specific neurologic agent used, the characteristics of the patient and the nature of the disease or condition for which the agent is being used. In addition, the concentration will depend upon whether the agent is being employed in a preventive or treatment capacity. Further, the stage of a particular disease or disorder, e.g., early vs. late Alzheimer's disease or Parkinson's disease, may dictate the optimal concentration of the agent.

In some embodiments, the dosage volume of the intranasal composition (applicable to nasal sprays or drops) range may be 0.015 ml-1.0 ml. In some embodiments, the dosage volume (applicable to nasal sprays or drops) range may be 0.03 ml-0.6 ml.

Generally, the treatment may be given in a single dose or multiple administrations, i.e., once, twice, three or more times daily over a period of time. For chronic disorders such as those diagnosed with, or at risk for, Alzheimer's disease, stroke or Parkinson's disease, the treatment may consist of at least one dose per day over an extended period of time. Post-stroke treatment may require more than one dose which may be administered several times over the course of a day, wherein this treatment regimen may encompass a week or more.

In some embodiments, a single dose comprises 100-400 mg of the intranasal composition disclosed here. In some embodiments, doses are administered once, twice, three, four, or five times daily, weekly, or monthly. In some embodiments, doses are administered 6-8 hours apart.

In some embodiments, the intranasal composition disclosed herein is effective to reduce or eliminate the symptoms of a degenerative neurological disease in a subject in need thereof. In some embodiments, the intranasal composition disclosed herein is effective to reduce or eliminate the symptoms of Parkinson's disease. In some embodiments, the intranasal composition disclosed herein is effective to increase grip strength, improve limb movement, and/or reduce tremor in a subject with Parkinson's disease. In some embodiments, the intranasal composition disclosed herein is effective to treat one or more of insomnia, neurological diseases or conditions, brain cancer, and inflammation in the brain.

### EXAMPLES

### Example 1—Formulations

### Nasal Spray Formulation 1

| Ingredients | % w/w (g) |
|---|---|
| Carnosine (S212) | 5% |
| Carboxymethyl cellulose (CMC) | 1%, 1.5%, 2% |
| P80 | 0.025% |
| Potassium sorbate | 0.12% |
| EDTA | 0.01% |

The nasal spray of Formulation 1 was made as follows. S212 was first dissolved in water to obtain a clear solution. Then P80 was added to form a S212 micelle preparation. Then CMC was added and mixed well to maintain the viscosity of formulation. Potassium sorbate was then added to serve as preservative and EDTA was added to as chelating agent to maintain the chemical stability of the formulation.

Nasal Spray Formulation 2 (with Cyclodextrin)

| Ingredients | % w/w (g) |
|---|---|
| Carnosine S212 | 5% |
| CMC | 1%, 2% |
| P80 | 0.025% |
| Potassium sorbate | 0.12% |
| EDTA | 0.01% |
| CD (Cavamax ® W8) | 5% |

Nasal Spray Formulation 3 (with Cyclodextrin)

| Ingredients | % w/w (g) |
|---|---|
| Carnosine S212 | 10% |
| CMC | 1%, 2% |
| P80 | 0.025% |
| Potassium sorbate | 0.12% |
| EDTA | 0.01% |
| CD (Cavitron ™ W7 HP5) | 5% |

The nasal spray of Formulations 2 and 3 was made according to Formulation 1, but Cyclodextrin (CD) was incorporated into new formulations with the same concentrations of constituents as the previous formulation. Specifically, carnosine was first dissolved in water to obtain a clear solution. Cyclodextrin was then added to mix and complex with carnosine under stirring to obtain a relatively clear cyclodextrin complex solution. Then P80 was added to form a hybrid micelle and cyclodextrin complex preparation. Then CMC was added and mixed well to maintain the viscosity of formulation. Potassium sorbate was then added to serve as preservative and EDTA was added to as chelating agent to maintain the chemical stability of the formulation.

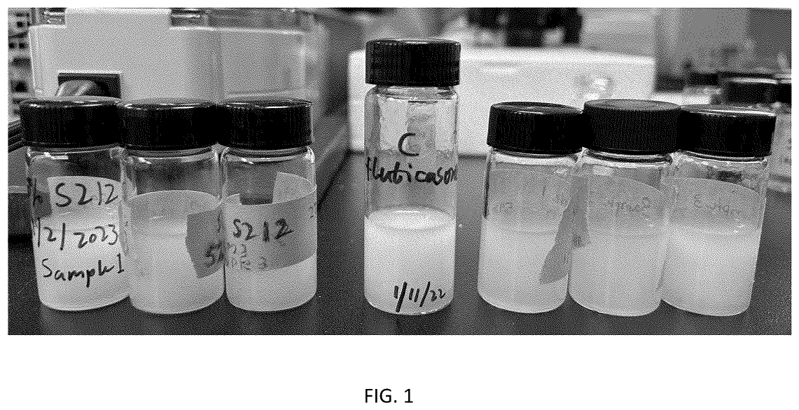

Formulations 1, 2, and 3 can be seen in FIG. 1 as a milky liquid, having concentrations of carnosine from 5% to 15% w/w.

### UV Absorbance Measurements

The absorbance of S212 solutions were measured using a spectrophotometer at wavelengths of 220 nm and 254 nm at concentrations of 1 mg/mL, 0.1 mg/mL, and 0.01 mg/mL.

| | Concentrations | | | | | |
|---|---|---|---|---|---|---|
| | 1 mg/mL | | 0.1 mg/mL | | 0.01 mg/mL | |
| | Wavelengths | | | | | |
| | 254 nm | 220 nm | 254 nm | 220 nm | 254 nm | 220 nm |
| Absorbance | 0.0023 | 2.4648 | 0.0092 | 1.0392 | 0.0014 | 0.1250 |
| Values | 0.0024 | 2.5923 | 0.0092 | 1.0407 | 0.0015 | 0.1253 |
| | 0.0023 | 2.4648 | 0.0092 | 1.0407 | 0.0016 | 0.1252 |

The absorbance values show increasing absorbance at 220 nm as concentrations increase from 0.01 mg/mL, to 0.1 mg/mL, to 1 mg/mL carnosine solution complezed with cyclodextrin.

### Two-Component Compatibility and Stability Studies by HPLC

Stability tests were conducted with eight samples consisting of S212 and one excipient; the samples were split into two groups of four. All samples contained 0.3 g of S212 each (10% of total concentration). Each group contained a sample comprising of one of the following: 3 g of CMC (1.5%; samples 1 and 2); 0.75 g of p80 (1%; samples 3 and 4); 3 g of potassium sorbate (12%; samples 5 and 6); 0.3 g EDTA (1%; samples 7 and 8). Samples 1, 3, 5, and 7 were stored at 25° C. and samples 2, 4, 6, and 8 were incubated at 40° C.; both groups remained undisturbed for more than a month and the 40° C. group was analyzed using HPLC. Each sample was diluted to achieve a 1 ug/mL concentration of S212 and placed in 2 mL HPLC vials. The absorbance graphs at 280 nm displayed eight minor peaks alongside a major peak, which resulted from detection of S212. Results highlight the possibility of impurities present, but roughly corresponded well with HPLC analysis conducted on S212 solutions, indicating stability and minimal effects of degradation at higher temperatures.

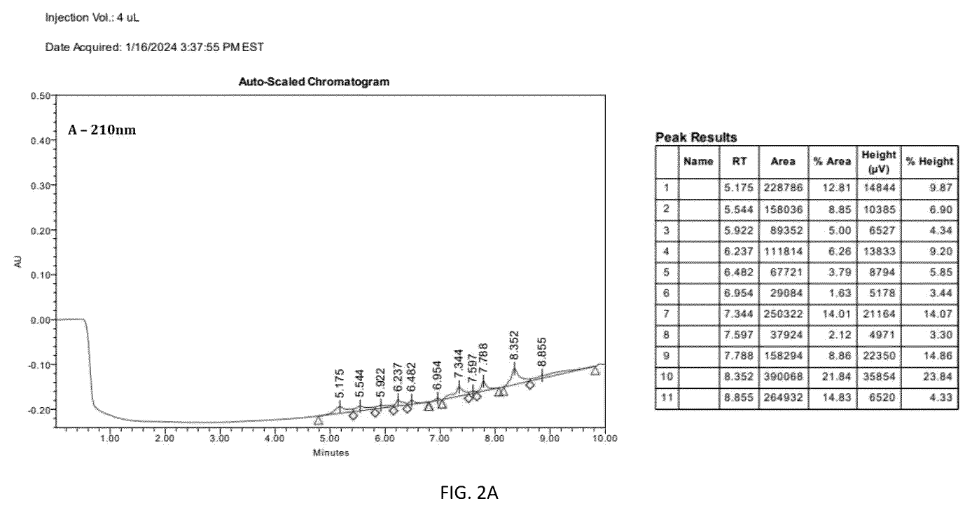

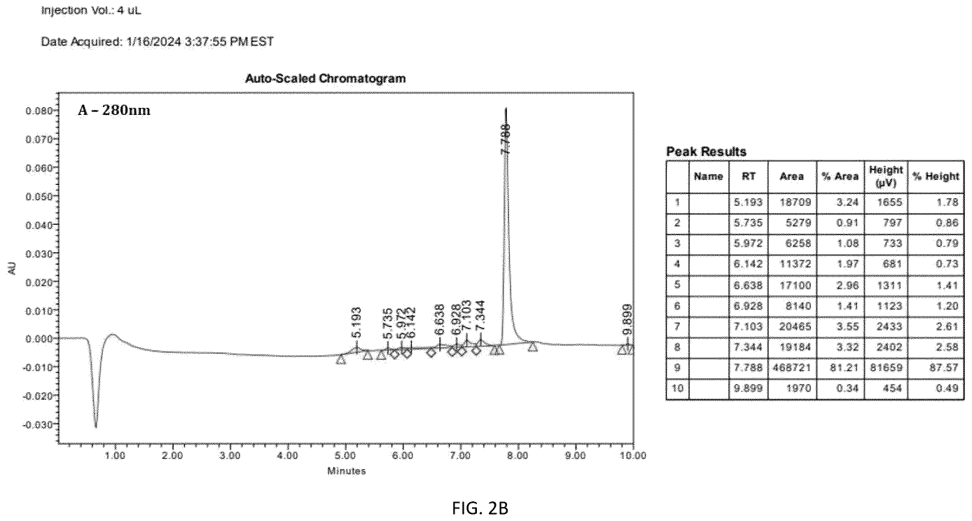

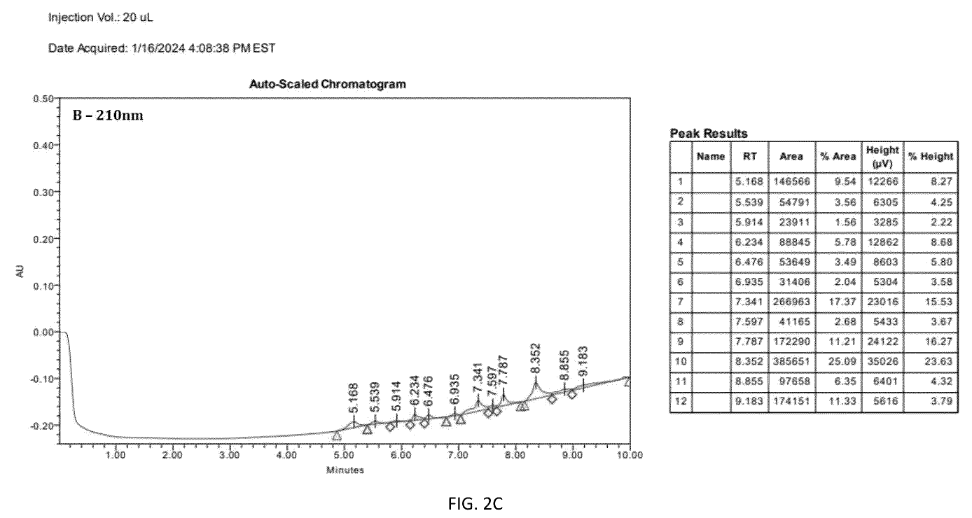

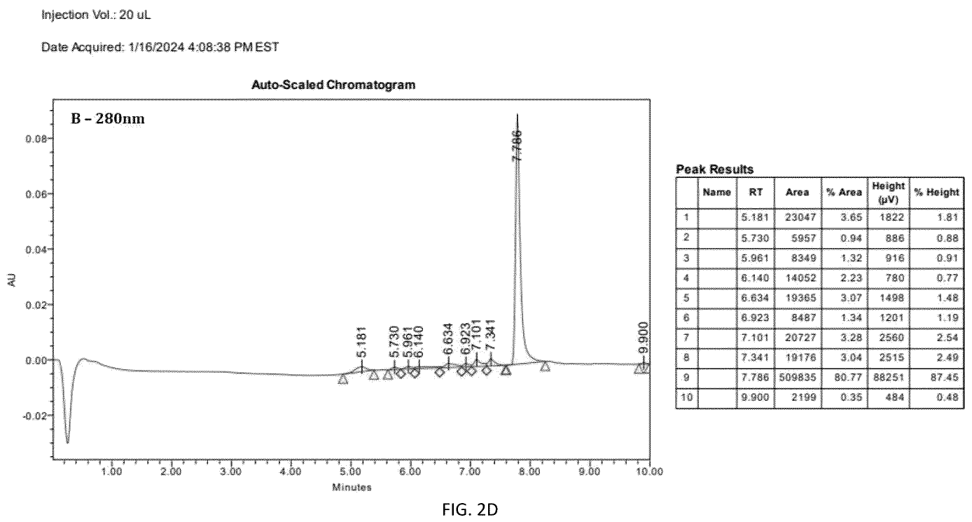

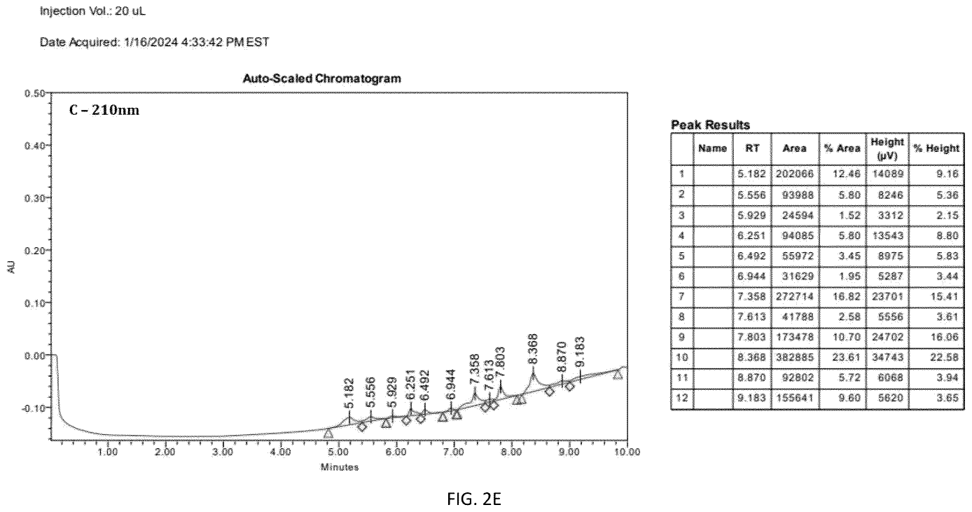

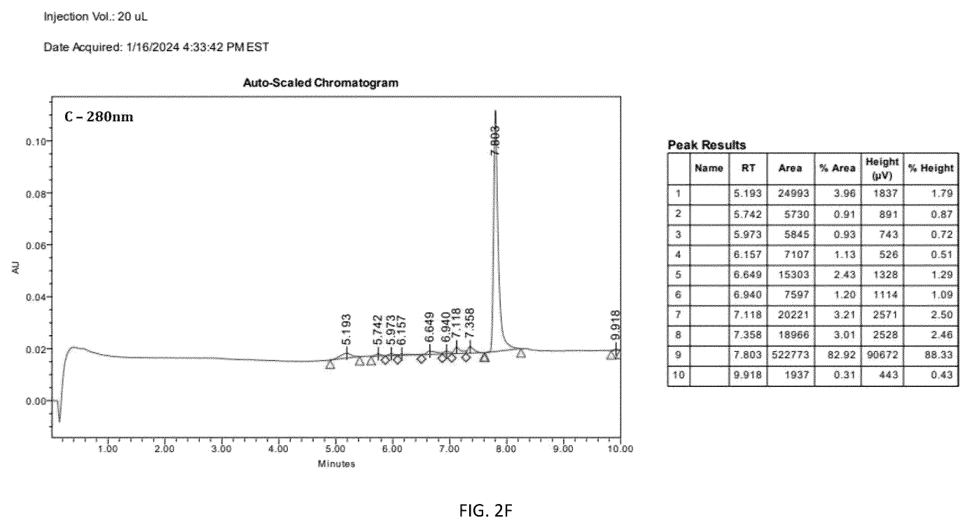

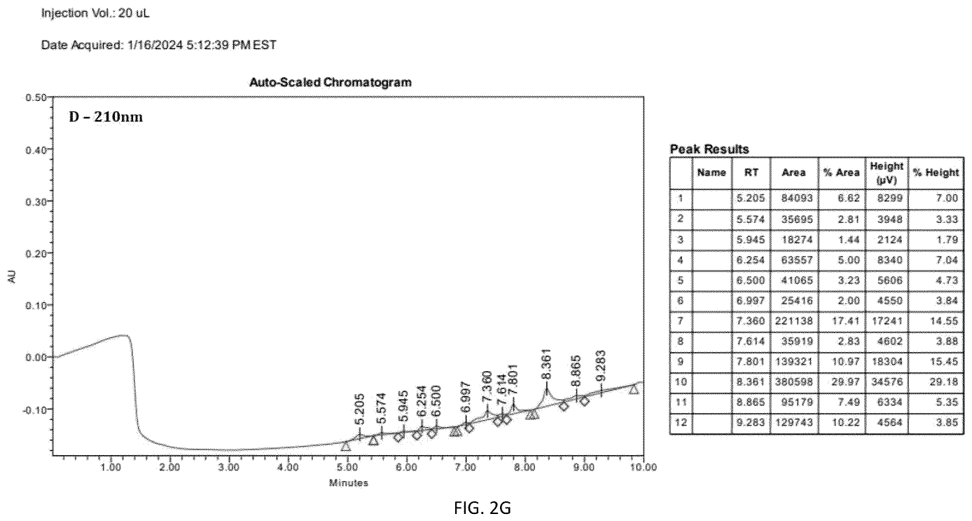

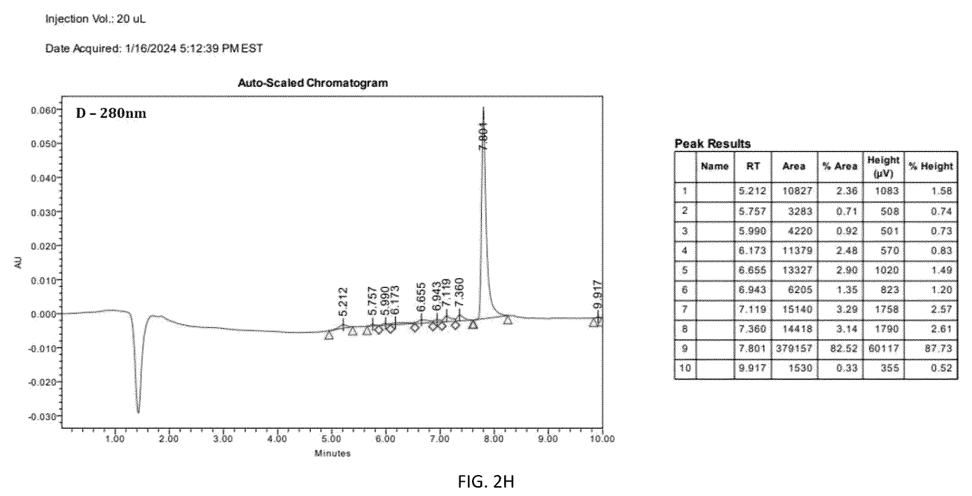

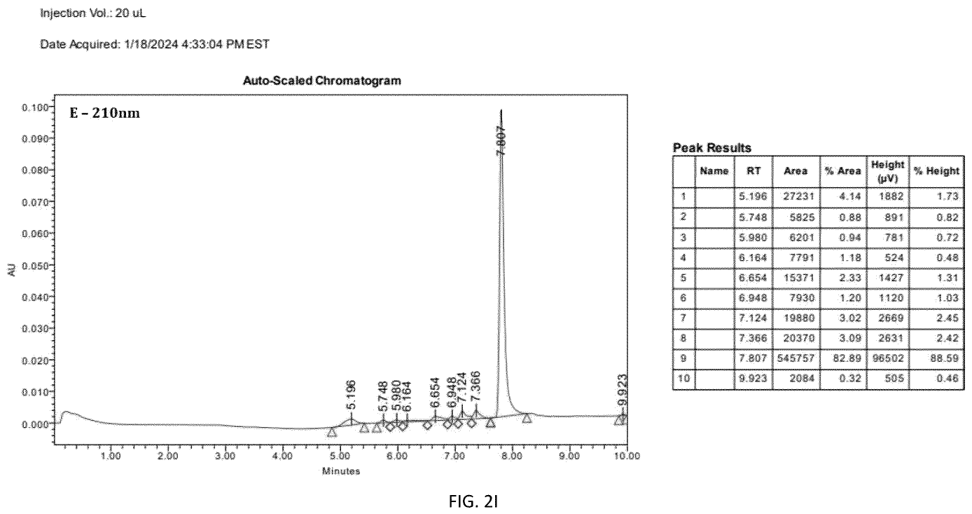

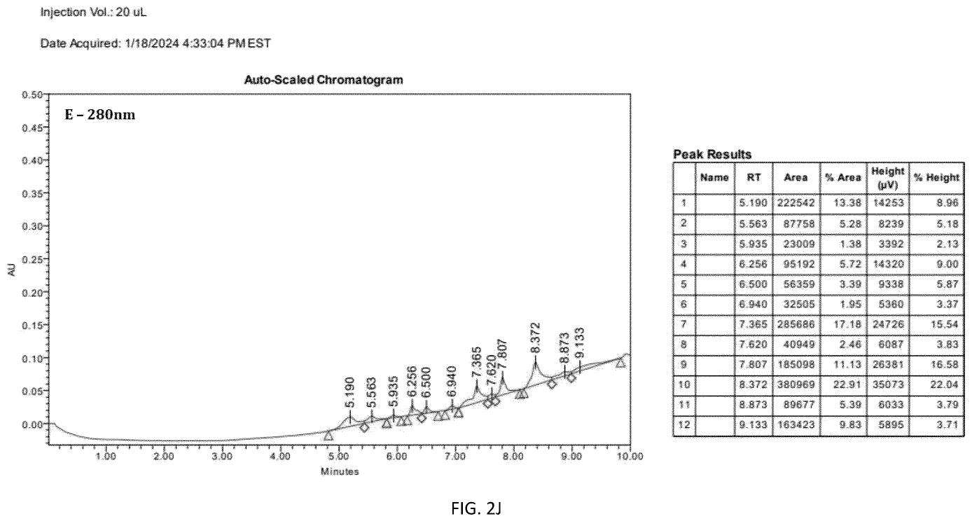

HPLC Chromatograms of 1 ug/mL S212 solutions and components after storage at 40° C. is shown in FIG. 2: Solution with only S212 (FIG. 2A/2B); solution containing MCG591p (sodium salt CMC) (FIG. 2B/2C, Sample 2); solution containing p80 (FIG. 2E/2F, Sample 4); solution with potassium sorbate (FIG. 2G/2H, Sample 6); and solution with EDTA (FIG. 2I/2J, Sample 8).

Stability Studies: HPLC Analysis of Nasal Spray Mixture without API

HPLC analysis conducted on a mixture comprising of CMC, p80, potassium sorbate, EDTA, and Cyclodextrin (CAVAMAX®) without API.

### PH Level of Spray Formulation

The pH levels of six samples were measured based on carnosine concentrations and compared to the Fluticasone. All samples, aside from Fluticasone, contained 5% S212; 1%, 1.5%, or 2% CMC; 0.025% p80; 0.12% potassium sorbate; and 0.01% EDTA. Two samples contained 1% CMC and 5 g of CD; one sample contained 2% CMC and 5 g of CD. pH of S212 Formulations and Fluticasone

| | | 1% | 1.5% | 2% |
|---|---|---|---|---|
| Sample | Fluticasone | carnosine | carnosine | carnosine |
| pH | 6.19 | 8.31 | 8.30 | 8.25 |
| pH with | | 8.25 | | 8.26 |
| CD | | 8.28 | | |

### Particle Size Measurements

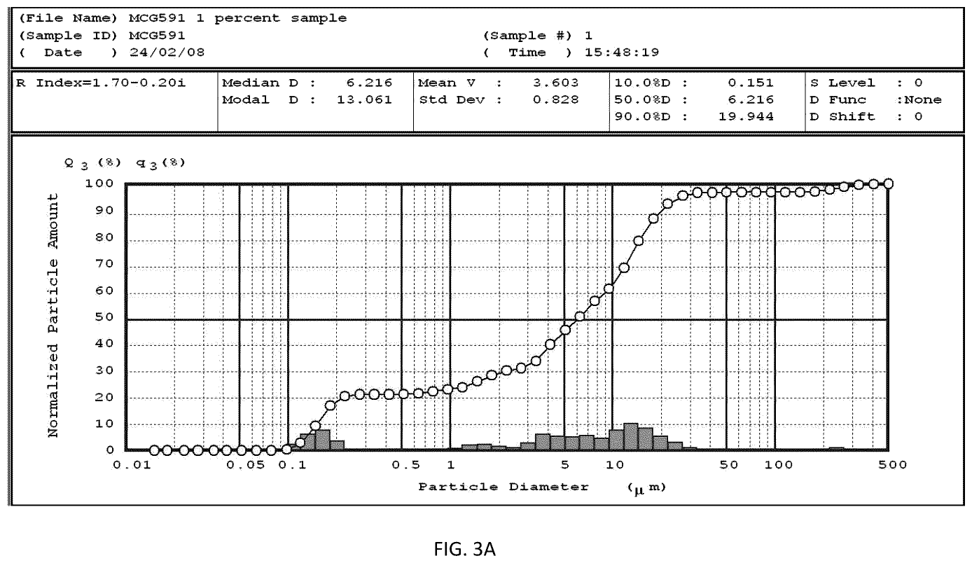

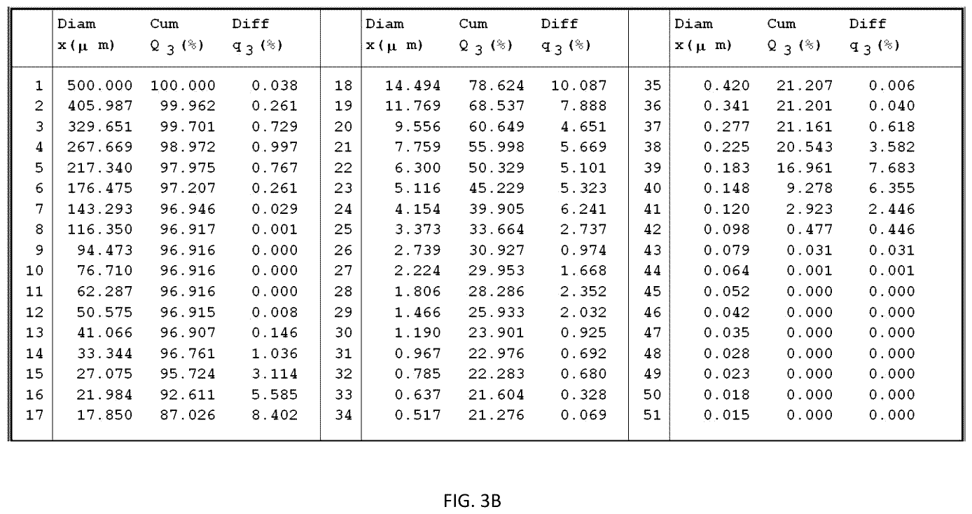

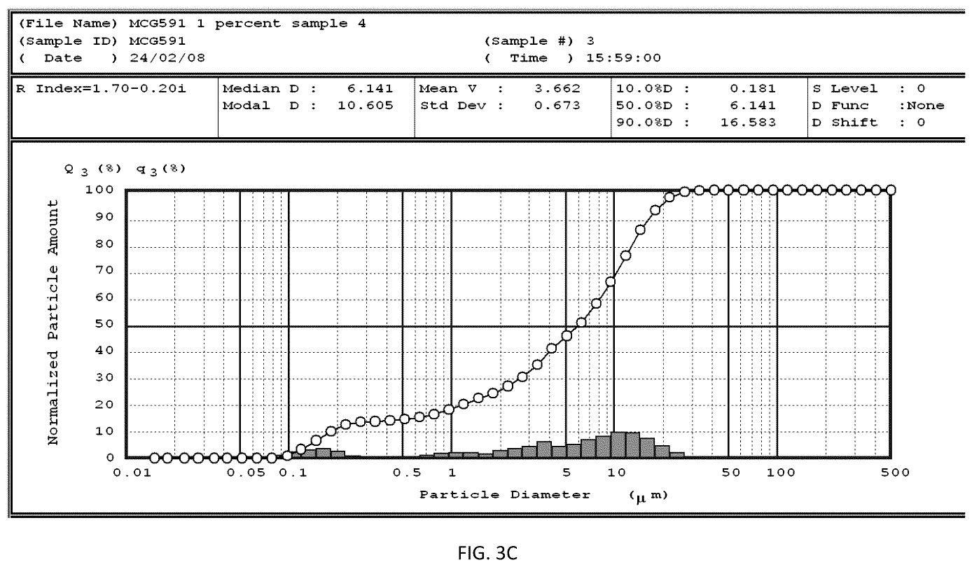

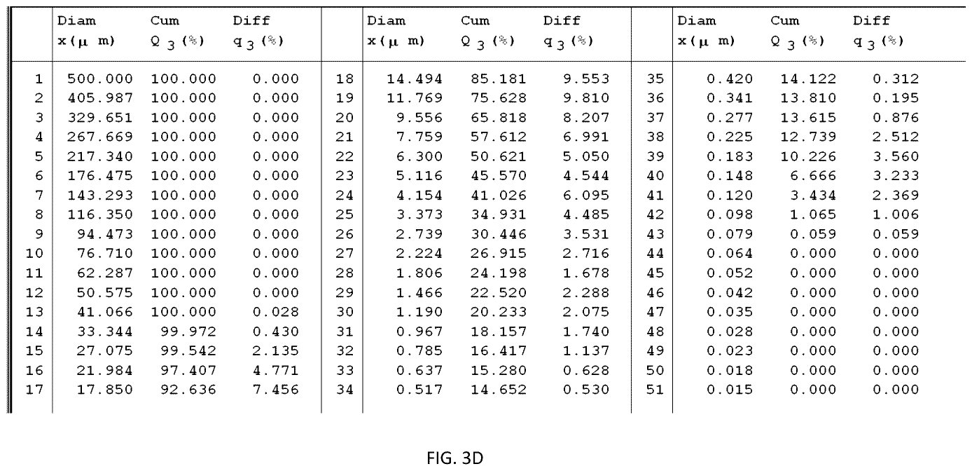

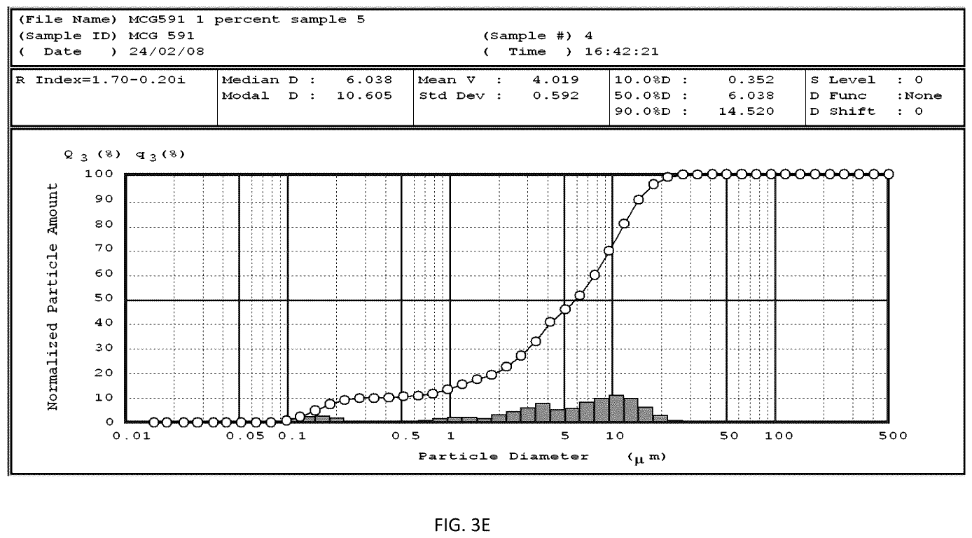

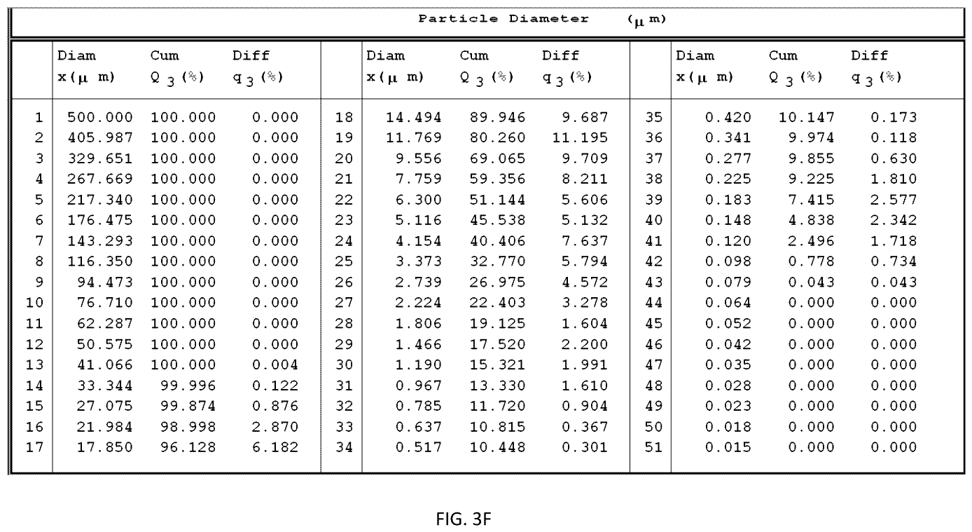

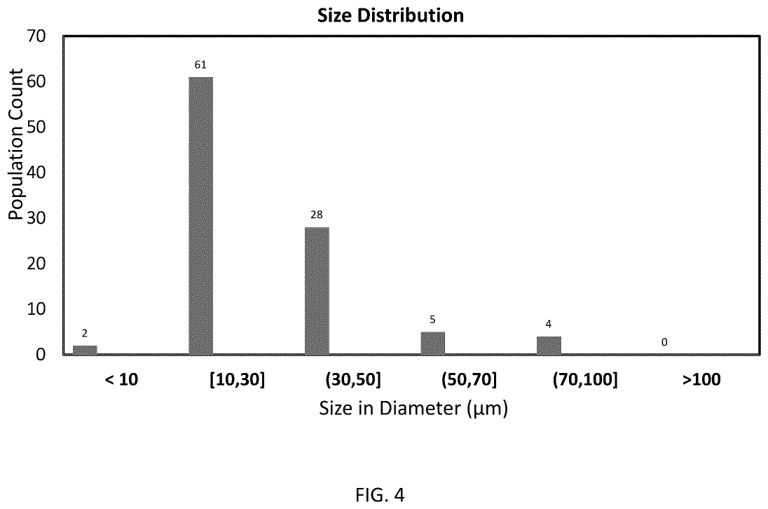

Particle size measurements and analysis were conducted with the SHIMADZU SALD-7001 Laser Diffraction Particle Size Analyzer and confirmed with measurements under a light microscope at 1000× total magnification. Data showing laser diffraction particle size analysis is shown in FIG. 3A-F.

Percent distribution of particle sizes measured under a light microscope at 1000× total magnification

| Percent distributions | 10% D | 50% D | 90% D |
|---|---|---|---|
| Particle size (μm) | 14 | 25 | 48 |

### Example 2—Neural Cell Gene Expression

### Carnosine Stock:

A stock of Carnosine was made at 884 mM-884 000 uM, it is “10×”, 20% mass/volume in a special media. This medium and all additives are from Stemcell Technologies and is composed of BrainPhys™ Neuronal Medium (Catalog #05795), with NeuroCult™ SM1 Neuronal Supplement (Catalog #05711), N2 Supplement-A (Catalog #07152), Human Recombinant BDNF (Catalog #78005), Human Recombinant GDNF (Catalog #78058), Dibutyryl-CAMP (Catalog #73882), Ascorbic Acid (Catalog #72132).

### Cell Differentiation and Cell Culture:

Astrocytes and Forebrain neurons were differentiated from iPSC-Derived Neural Progenitor Cells (catalog #200-0620) according to vendor protocol (Stemcell Technologies).

Microglia were differentiated from iPSC-Derived Healthy control human iPSC line SCTi003-A (Catalog #200-0510) according to vendor protocol (Stemcell Technologies).

The differentiated Astrocytes/Forebrain neurons/Microglia were co-cultured in BrainPhys™ Neuronal Medium complete medium which consists of BrainPhys™ Neuronal Medium (Catalog #05795), with NeuroCult™ SM1 Neuronal Supplement (Catalog #05711), N2 Supplement-A (Catalog #07152), Human Recombinant BDNF (Catalog #78005), Human Recombinant GDNF (Catalog #78058), Dibutyryl-cAMP (Catalog #73882), Ascorbic Acid (Catalog #72132).

### Experiment:

The experiment contains 4 different cell culture sets: 1. co-culture of Astrocytes/Forebrain neurons/Microglia; 2. Microglia alone; 3. Astrocytes alone; 4. Forebrain neurons alone.

The co-culture of Astrocytes/Forebrain neurons/Microglia as well as the 3 types of cells cultured alone were all grown as: 1) half grown in BrainPhys™ Neuronal Medium complete medium with 1 uM final Amyloid 42-1 Inactive peptide (Catalog #Ab120481, vendor #Abcam) for 24 h, and 2) half in BrainPhys™ Neuronal Medium complete medium with 1 uM final Amyloid Plaque 1-42 Active peptide (Catalog #Ab 120301, vendor #Abcam), with TNF-alpha at 100 ng/mL (Catalog #Ab259410, vendor #Abcam) and with IFN-gamma at 100 ng/ml (catalog #Ab259377, vendor #Abcam) for 24 h.

After the 24 h, the two conditions were each split in 4 different groups: 1. Negative control (0 mM carnosine); 2. Carnosine was added at final concentration of 44.2 mM; 3. Carnosine was added at final concentration of 0.844 mM; 4. Carnosine was added at final concentration of 0.00884 mM.

After 48 h of carnosine dosing, the media were discarded, the cells were lysed using lysis buffer (RLA competed with beta-mercaptoethanol) from a kit of total RNA extraction (SV Total RNA Isolation System) as specified by the vendor (Promega). Each condition, each replicate is extracted as a unique RNA sample. Each condition is done in multi-replicates.

After total RNA extraction, RNA is quantified and checked for quality by measuring the Optical Density of each individual RNA sample.

A reverse transcription is performed on each individual RNA sample at 80 ng of total RNA using the SuperScript™ IV VILO™ Master Mix from vendor ThermoFisher Scientific following manufacturer instructions to produce a cDNA equivalent to each RNA sample in a machine used as a thermocycler.

After reverse transcription, each sample is diluted 1:1 with molecular biology grade water. After dilution of the cDNA samples, a real-time PCR is performed on each cDNA sample (4 ng) set as multiplex, meaning two Taqmans are used simultaneously for each sample using a real-time PCR machine; one Taqman is the Eukaryotic 18S rRNA Endogenous Control the other Taqman is the target gene. The real-time PCR data is run as comparative Ct (delta delta Ct).

There is a new real-time PCR for each couple Eukaryotic 18S rRNA Endogenous Control the other Taqman is the target gene versus target gene Taqman. All target genes Taqmans are for human genes.

Target genes were selected for genes know to play a role or suggested to play a role in microglia or astrocyte or neurons during the course of Alzheimer's disease. The different couples are as follows:

Eukaryotic 18S rRNA Endogenous Control Taqman (VIC™/MGB probe, primer limited, vendor #ThermoFisher Scientific, catalog #4319413E) and h TGF-beta 1 (vendor #ThermoFisher Scientific, Assay ID #Hs00998133_m1)

Eukaryotic 18S rRNA Endogenous Control Taqman (VIC™/MGB probe, primer limited, vendor #ThermoFisher Scientific, catalog #4319413E) and h MMP-9 (vendor #ThermoFisher Scientific, Assay ID #Hs00957562_m1)

Eukaryotic 18S rRNA Endogenous Control Taqman (VIC™/MGB probe, primer limited, vendor #ThermoFisher Scientific, catalog #4319413E) and h IDE (vendor #ThermoFisher Scientific, Assay ID #Hs00610452_m1)

Eukaryotic 18S rRNA Endogenous Control Taqman (VIC™/MGB probe, primer limited, vendor #ThermoFisher Scientific, catalog #4319413E) and h ECE-1 (vendor #ThermoFisher Scientific, Assay ID #Hs01043735_m1)

Eukaryotic 18S rRNA Endogenous Control Taqman (VIC™/MGB probe, primer limited, vendor #ThermoFisher Scientific, catalog #4319413E) and h GDNF (vendor #ThermoFisher Scientific, Assay ID #Hs01931883_s1) limited, vendor #ThermoFisher Scientific, catalog #4319413E) and h ET-1 (vendor #ThermoFisher Scientific, Assay ID #Hs00174961_m1)

Eukaryotic 18S rRNA Endogenous Control Taqman (VIC™/MGB probe, primer limited, vendor #ThermoFisher Scientific, catalog #4319413E) and h IL-33 (vendor #ThermoFisher Scientific, Assay ID #Hs04931857_m1)

Eukaryotic 18S rRNA Endogenous Control Taqman (VIC™/MGB probe, primer limited, vendor #ThermoFisher Scientific, catalog #4319413E) and h CXCR4 (vendor #ThermoFisher Scientific, Assay ID #Hs00237052_m1)

Eukaryotic 18S rRNA Endogenous Control Taqman (VIC™/MGB probe, primer limited, vendor #ThermoFisher Scientific, catalog #4319413E) and h SDF-1 (vendor #ThermoFisher Scientific, Assay ID #Hs03676656_mH)

Eukaryotic 18S rRNA Endogenous Control Taqman (VIC™/MGB probe, primer limited, vendor #ThermoFisher Scientific, catalog #4319413E) and h SIRT-1 (vendor #ThermoFisher Scientific, Assay ID #Hs01009006_m1)

Eukaryotic 18S rRNA Endogenous Control Taqman (VIC™/MGB probe, primer limited, vendor #ThermoFisher Scientific, catalog #4319413E) and h IL-6 (vendor #ThermoFisher Scientific, Assay ID #Hs00174131_m1)

Eukaryotic 18S rRNA Endogenous Control Taqman (VIC™/MGB probe, primer limited, vendor #ThermoFisher Scientific, catalog #4319413E) and h CXCL10 (vendor #ThermoFisher Scientific, Assay ID #Hs00171042_m1) limited, vendor #ThermoFisher Scientific, catalog #4319413E) and h IL-10 (vendor #ThermoFisher Scientific, Assay ID #Hs00961622_m1)

t TEST (sample compared to control) was performed for the obtained data, with p value equal of less than 0.05 being considered as specific.

MMP-9, IDE, ECE-1 are proteases described as cleaving Amyloid Plaque.

BDNF is a neuronal growth factor.

ET-1 is a growth factor for Oligodendrocytes. Oligodendrocytes feed and protect neurons.

IL-33 is a described as decreasing Alzheimer's disease-like pathology, including decreasing cognitive decline.

SDF-1 (CXCL12) is the ligand of CXCR4.

SIRT-1 is a survival factor.

IL-6, CXCL10 are cytokines that increase inflammation.

IL-10 is an interleukin that reduces inflammation.

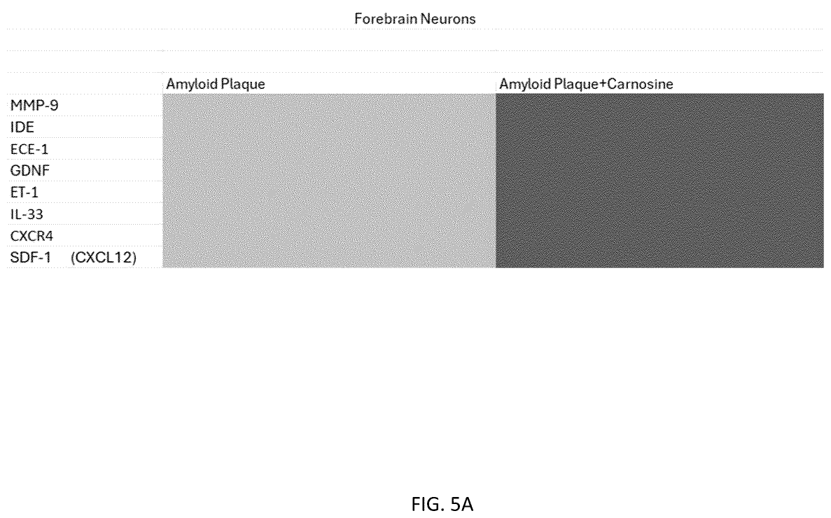

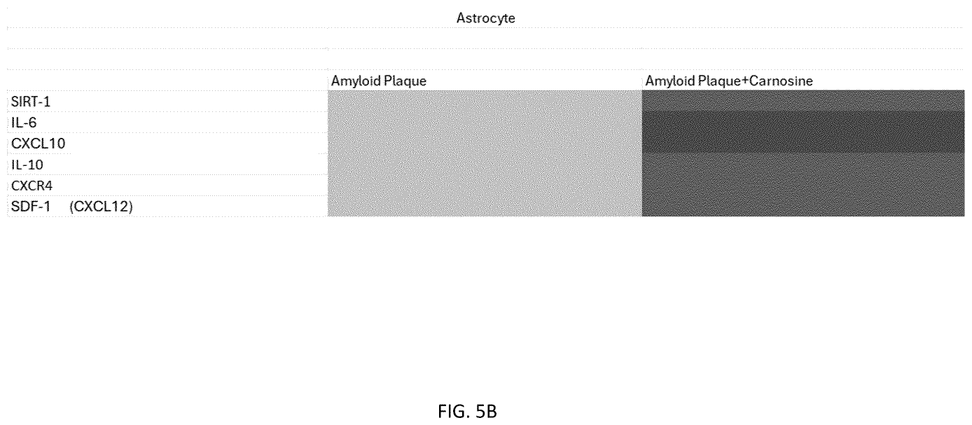

As shown in FIGS. 5A and 5B, green indicates upregulation and red indicate downregulation of the respective gene expression.

### Example 3—Parkinson Disease Mouse Model Study

Summary: The efficacy of carnosine (S212 nasal spray) via intranasal drug delivery was tested for 10 days (once a day) in subacute Parkinson's disease (PD) mouse model induced by MPTP. After prescreening of the Pole test and the Rotarod test, the qualified mice were randomly divided into 6 groups according to body weight, with 10 mice in each group, which was: (I) Control group, (II) Model group, (III) Positive drug group (100 mg/kg), (IV) S212 nasal spray on high dose group (15 μL/mouse), (V) S212 nasal spray on middle dose group (10 μL/mouse), (VI) S212 nasal spray on low dose group (5 μL/mouse). Subsequently, the mice except for the Control group were induced by injection of MPTP (ip., 25 mg/kg, 5 mL/kg) once a day for 10 days. The treatment of the test article was taken after MPTP every day. The first day of inducing was recorded as Day1, the measurement of the limbing Pole test, the Rotarod test and Grip strength test were be done on Day6 and Day 10. The brain of all mice was extracted and fixed in 4% paraformaldehyde solution.

Results: (1) the general condition of mice: The mice showed abnormal symptom such as decreased activity, erect hair and tremor after injection of MPTP, and recovered gradually 1 hour after injection. These abnormal symptom can be little reduced after medication treatment, but it can not completely disappear. In addition, the mice were hyperexcitability after the positive drug 100 mg/kg metoba was administered, and two mice of the Medopa group died (respectively at Day6 and Day7). (2) the Rotarod test: 100 mg/kg Medoba and nasal spray S212 (5, 10, 15 μL/mouse) for 10 days of administration can improve the limb movement injury induced by MPTP in subacute PD model mice, showed as prolonging the Latency of the first fall and reducing the fall frequency of subacute PD mice. However, the nasal spray S212 (5, 10, 15 μL/mouse) had no obvious dose-dependent relationship, and were slightly weaker than the 100 mg/kg Medoba. (3) the limbing Pole test: 100 mg/kg Medoba and nasal spray S212 (5, 10, 15 μL/mouse) for 10 days of administration can improve the limb movement injury induced by MPTP in subacute PD model mice, showed as shortening the time of pole climbing and increasing the gait score of the mice. However, the nasal spray S212 (5, 10, 15 μL/mouse) had no obvious dose-dependent relationship, and were slightly weaker than the 100 mg/kg Medoba. (4) the Grip strength test: the muscle tension and grip strength of mice in the model group decreased (P<0.01 vs. Control group). The 100 mg/kg Medoba significantly improved the grip strength of PD mice (P<0.001 vs Model group). The S212 (5, 10, 15 μL/mouse) significantly increased the grip strength of PD model mice, but there was no obvious dose-dependent relationship. (5) the body weight of mice: he positive drug 100 mg/kg Medoba and test article S212 (5, 10, 15 μL/mouse) had no significant effect on the body weight of PD mice.

Conclusion: In this study, S212 nasal spray (5, 10, 15 μL/mouse) via intranasal drug delivery for 10 days (once a day) can improve the limb movement injury of subacute PD model mice induced by MPTP, but had no obvious dose-dependent relationship.

### Compound and Preparation

Test materials: The formulation used in the testing of the animal model was 0.5% Sodium CMC; 20% carnosine; and 5% gamma-cyclodextrin; and 0.025% Polysorbate 80.

Positive control drug: Name: Dobasic Hydrazide Tablets (Medopa®); Manufacturer: Shanghai Roche Pharmaceutical Co.; Description: Reddish tablets with coloring agent; Batch number: YT0941; Specification: 250 mg/tablet; Storage conditions: Shield from light, seal and store in a cool dry place.

PD model inducing agent: Name: 1-methyl-4-phenyl-1,2,3,6-tetrahydropyridine hydrochloride (MPTP hydrochloride); Manufacturer: Sigma-Aldrich; Description: white or off-white powder; Molecular Formula: C12H15N—HCl; Molecular weight: 209.72; Specification: 100 mg/vial; CAS-No: 23007-85-4; Storage condition: Protected from light, room temperature.

Negative control agent: Name: Sodium chloride injection; Manufacturer: Zhejiang Tianrui Pharmaceutical Co., Ltd.; Description: Colorless transparent liquid; Batch number: 124040801; Specification: 250 ml/vial; Storage conditions: RT.

Negative control agent: Name: Carboxymethyl cellulose sodium Manufacturer: Shanghai Aladdin Biochemical Technology Co., Ltd Description: White fibrous or granular powder; Batch number: E2411161; Specification: 500 g/bottle; Storage conditions: Room Temperature.

### Equipment Used:

Name: Rotating rod fatigue meter; Manufacturer: Huabei Zhenghua Biological Instrument Co. Type: YLS-4C.

Name: Mouse Grip Tester; Manufacturer: Jinan Yiyan Technology Development Co.; Type: YLS-13A.

Name: Mouse pole climbing instrument; Specification: Wooden pole (1 cm in diameter, 50 cm in height) with small ball at the top; Source: Self-made.

### Animals and Husbandry:

Animals: Strain and Species: C57BL/6J mice; Grade: SPF; Gender: Male; Source: Zhejiang Vital River Laboratory Animal Technology Co., Ltd.; Number of animals: 72 order; Age at animals arrive: 7˜8 w; Upon arrival at the animal facility animals were housed 5˜6 mouse per cage and acclimated for three to seven days.

Environment: Environmental controls for the animal room was set to maintain a temperature of 20˜25° C., humidity of 40˜70%, and a 12-hour light/12-hour dark cycle. The 12-hour dark cycle was temporarily interrupted to accommodate study procedures.

Food and water: SPF Mice Growth Breeding Feed (Jiangsu Xietong Pharmaceutical Bio-engineering Co., Ltd.) provided ad libitum throughout the in-life part of the study. Reverse Osmosis water was available ad libitum.

Animal selection: Animals used in this study were selected based on overall health and acclimation to caging.

### Methods

Adaptive training and prescreening of mice: The mice were trained and screening before modeling, including Rotarod test and Pole test, once a day for three consecutive days. The training of Rotarod test daily was after the Pole test.

The training of Pole test: Mice were placed head-down on the small ball at the top of a wooden pole (1 cm in diameter and 50 cm in height), and climbed down naturally along the pole. Gait score and time taking to the bottom were recorded during the climbing process. Mice were trained three times a day for 3 days. Pole climbing gait was scored according to the following criteria:
5 points: Crawl down using four limbs, step by step in coordination.
4 points: Crawl down step by step, but with sliding slightly of hind legs.
3 points: Crawl with sliding after half the distance, but can hold the pole.
2 points: Crawl with sliding significantly after half the distance.
1 point: Fall from the pole after climbing half the distance, and cannot grasp the pole.

Pole climbing test screening: the unqualified animals that could not climb down smoothly or stayed on the pole for too long were eliminated after the third day of pole climbing training.

The training of Rotarod test: The mice were placed on the rotarod, and climb along with it after turning on. The rotation speed and training time during these 3 days are different. It was 14 rpm for 6 min on the first day, 18 rpm for 4 min on the second day, and 30 rpm for 5 min on the third day. The mice falling from the rotarod were eliminated on the third day.

### Animal Groups and Drugs:

Groups were formed according to the following table:

| | | | | | | Dosing route, |
|---|---|---|---|---|---|---|
| | | Animal | Dose | Dose volume | Concentration | frequency & |
| Group | Treatment | Number | (mg/mouse) | (μL/mouse) | (mg/mL) | period |
| 1 | Control | 10 | — | 15 | — | Intranasal drug |
| 2 | Model | 10 | — | 15 | — | delivery, |
| | | | | | | Qdx10 days |
| 3 | Medopa | 10 | 100 mg/kg | 10 mL/kg | 10 | i.g., Qdx10 days |
| 4 | S212 nasal | 10 | 1.5 | 15 | 100 | Intranasal drug |
| | spray, high | | | | | delivery, |
| | dose | | | | | Qdx10 days |
| 5 | S212 nasal | 10 | 1 | 10 | 100 | |
| | spray, | | | | | |
| | middle | | | | | |
| | dose | | | | | |
| 6 | S212 nasal | 10 | 0.5 | 5 | 100 | |
| | spray, low | | | | | |
| | dose | | | | | |

### Drug Configuration

Preparation of positive drugs: To take 1 tablet of Medoba, grind, add 25 ml of 0.5% CMC-Na, mix thoroughly and evenly.

Preparation of MPTP (25 mg/kg, 5 mL/kg): To weigh 50 mg MPTP hydrochloride, dissolve with sodium chloride injection, constant volume to 10 mL, prepare before use, store away from light.

Method: After adaptive training and prescreening, the qualified mice were randomly divided into 6 groups according to body weight, with 10 mice in each group, which was: (I) Control group, (II) Model group, (III) Positive drug group (100 mg/kg), (IV) S212 nasal spray on high dose group (15 μL/mouse), (V) S212 nasal spray on middle dose group (10 μL/mouse), (VI) S212 nasal spray on low dose group (5 μL/mouse). Subsequently, the mice except for the Control group were induced by injection of MPTP (ip., 25 mg/kg, 5 mL/kg) once a day for 10 days. The treatment of the test article was taken after MPTP everyday. The first day of inducing was recorded as Day1, the measurement of the Pole test, the Rotarod test and Grip strength test were be done on Day6 and Day10. The brain of all mice was extracted and fixed in 4% paraformaldehyde solution (to take the immunohistochemical staining of TH in substantia nigra densa).

Statistical analysis: The data were presented as Mean±SD, and the measured values that deviated from the mean by more than two standard deviations were excluded. T-test analysis was used for data analyzing. The statistical significance was considered when the P value is less than 0.05. The final data was plotted with GraphPad Prism software.

### Results

### The Effect of S212 Nasal Spray Via Intranasal Drug Delivery for 10 Days (Once a Day) on the General Condition in MPTP-Induced Subacute PD Model Mice

The mice showed abnormal symptom such as decreased activity, erect hair and tremor after injection of MPTP, and recovered gradually 1 hour after injection. These abnormal symptoms reduced after medication treatment, but do not completely disappear. In addition, the mice had hyperexcitability after the positive drug 100 mg/kg metoba was administered, and two mice of the Medopa group died (respectively at Day6 and Day7). No obvious abnormalities were found in the autopsy, and possible operational problems of the experimentors were ruled out.

### The Effect of S212 Nasal Spray Via Intranasal Drug Delivery for 10 Days (Once a Day) on the Rotarod Test in MPTP-Induced Subacute PD Model Mice

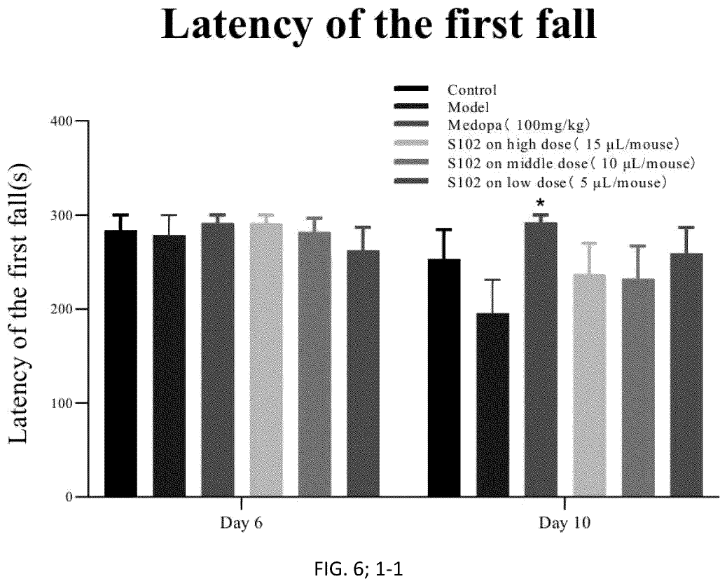

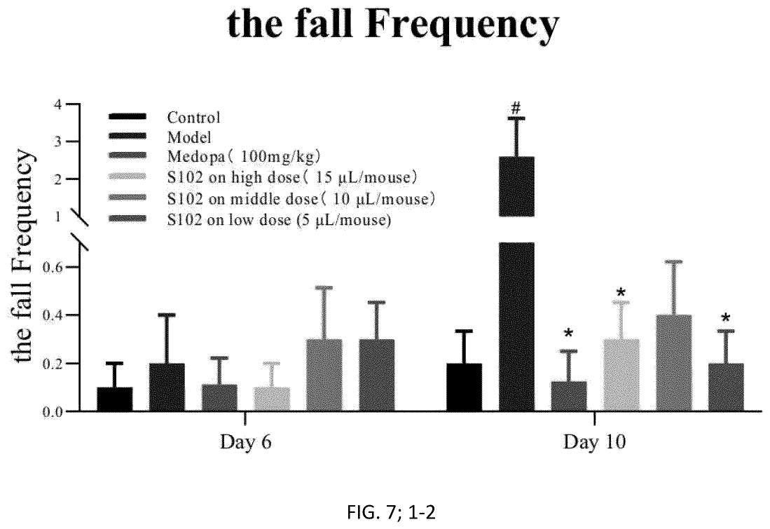

There are two main indexes of the Rotarod test: (1) the first time the mouse fell from the rod (the Latency of the first fall, seconds), and (2) the number of times each mouse falls from the Rotarod fall in 5 min (the fall frequency). These two indicators should be considered together when evaluating drug efficacy.
(1) The effect on the Latency of the first fall: According to the data in FIG. 6 (1-1) and Tab.1-1, a few mice in the Control group still fall from the rod at Day5 and Day10, but it was observed that they jumped with their own initiative, rather than falling with the poor coordination of their limbs. At Day10, the latency of the model group was significantly lower than that of the Control group, and it indicate that the mice's motor ability was impaired after 10 days of MPTP modeling. After continuous administration for 10 days, 100 mg/kg Medoba had a significant effect on prolonging the fall-latency of subacute PD mice (P<0.05). The S212 nasal spray (5, 10, 15 μL/mouse) showed a tendency to prolong the latency of the first fall, but there was no statistical difference due to the large individual differences, and there was no obvious dose-dependent relationship. And the S212 nasal spray (5, 10, 15 μL/mouse) were slightly weaker than the 100 mg/kg medoba.
(2) The effect on the fall frequency in 5 min: According to the data in FIG. 7 (1-2) and Tab. 1-2, at Day10, the fall frequency in 5 min of PD mice in model group were significantly increased compared with control group (P<0.05). After continuous administration for 10 days, 100 mg/kg Medoba could significantly reduce the fall frequency of PD mice (P<0.05 vs model group), and subject S212 (5, 10, 15 μL/mouse) could also significantly reduce fall frequency of PD mice, but there was no obvious dose-dependent relationship.

In summary, in the Rotarod test, 100 mg/kg Medoba and nasal spray S212 (5, 10, 15 L/mouse) for 10 days of administration can improve the limb movement injury induced by MPTP in subacute PD model mice, showed as prolonging the Latency of the first fall and reducing the fall frequency of subacute PD mice. However, the nasal spray S212 (5, 10, 15 μL/mouse) had no obvious dose-dependent relationship, and were slightly weaker than the 100 mg/kg Medoba.

TABLE 1-1
The effect of S212 nasal spray via intranasal drug delivery for
10 days (once a day) on the Latency of the first fall in the
Rotarod test in MPTP-induced subacute PD model mice(x ± sd)

| | | Latency of first fall (s) | |
|---|---|---|---|
| Group | N | Day 6 | Day 10 |
| Control | 10 | 283.8 ± 51.23 | 253.3 ± 98.53 |
| Model | 10 | 278.4 ± 68.31 | 195.5 ± 112.35 |
| Medopa (100 mg/kg) | 8 | 291.56 ± 25.33 | 292 ± 22.63* |
| S212 (15 μL/mouse) | 10 | 291.2 ± 27.83 | 237.3 ± 103.04 |
| S212 (10 μL/mouse) | 10 | 282 ± 45.69 | 232.1 ± 110.03 |
| S212 (5 μL/mouse) | 10 | 262.4 ± 77.7 | 259.4 ± 86.28 |

*P < 0.05 vs. Model;
two mice of the Medopa group died.

TABLE 1-2
The effect of S212 nasal spray via intranasal drug delivery
for 10 days (once a day) on the Frequency of the fall in the
Rotarod test in MPTP-induced subacute PD model mice(x ± sd)

Fall frequency

| Group | N | Day 6 | Day 10 |
|---|---|---|---|
| Control | 10 | 0.1 ± 0.32 | 0.2 ± 0.42 |
| Model | 10 | 0.2 ± 0.63 | 2.6 ± 3.24 # |
| Medopa (100 mg/kg) | 8 | 0.11 ± 0.33 | 0.13 ± 0.35* |
| S212 (15 μL/mouse) | 10 | 0.1 ± 0.32 | 0.3 ± 0.48* |
| S212 (10 μL/mouse) | 10 | 0.3 ± 0.67 | 0.4 ± 0.7 |
| S212 (5 μL/mouse) | 10 | 0.3 ± 0.48 | 0.2 ± 0.42* |

# P < 0.05 vs. Control,
*P < 0.05 vs. Model;
two mice of the Medopa group died.

### The Effect of S212 Nasal Spray Via Intranasal Drug Delivery for 10 Days (Once a Day) on the Climbing Pole Test in MPTP-Induced Subacute PD Model Mice

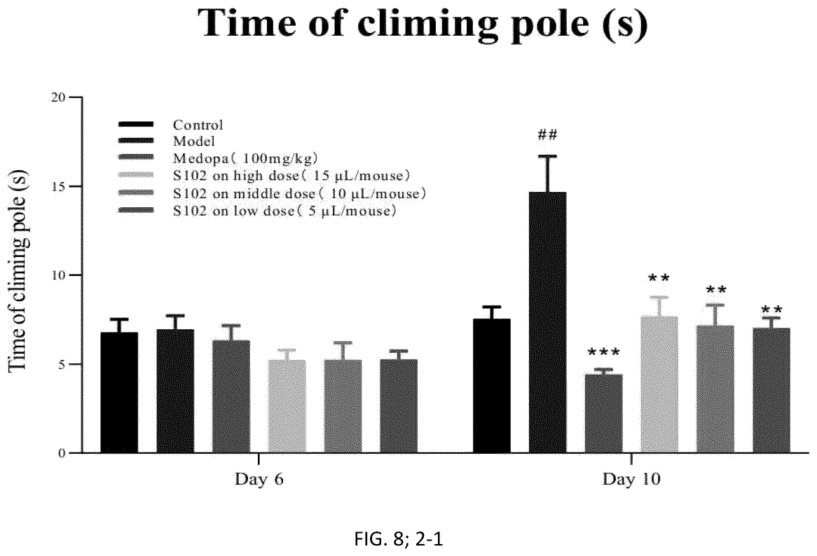

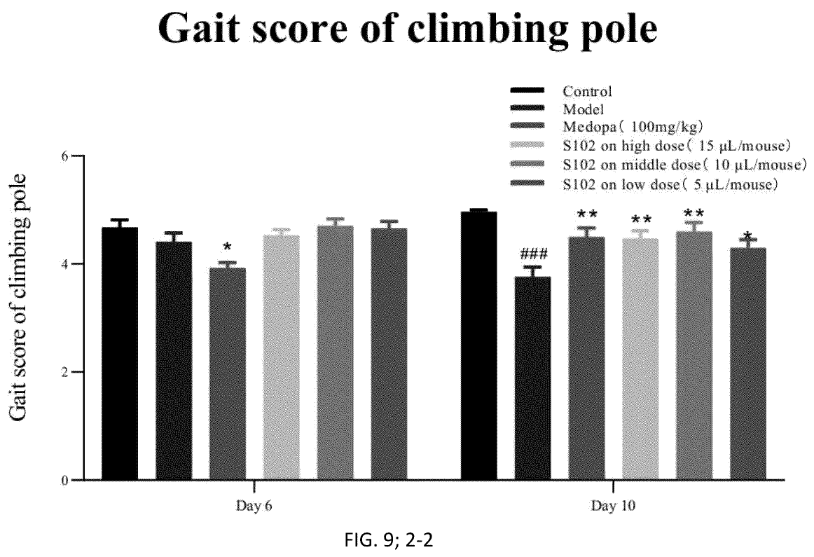

There are two main index of the climbing pole test: (1) the time required to climb from the top to the bottom of the pole (the climbing pole time), and (2) the gait score of climbing.
(1) The effect on the climbing pole time: According to the data in FIG. 8 (2-1) and Tab.2-1, compared with the Control group, the climbing time of PD mice in the Model group did not increase significantly on Day6, whereas it was significantly increase on the Day10 (P<0.01), suggesting that the PD mice had certain motor function damage after 10 days of modeling. After continuous administration for 10 days, 100 mg/kg Medoba and nasal spray S212 (5, 10, 15 μL/mouse) could significantly reduce the climbing pole time (P<0.01, P<0.001); However, the nasal spray S212 (5, 10, 15 μL/mouse) had no obvious dose-dependent relationship, and were slightly weaker than the 100 mg/kg Medoba.
(2) The effect on the gait score of climbing: According to the data in FIG. 9 (2-2) and Tab.2-2, the gait score of PD mice in the Model group decreased significantly compared with that in the control group after 10 days of modeling, indicating that the grasp-ability of PD mice was weakened, and leaded to unstable gait and sliding. After administration for 10 days, 100 mg/kg Medoba and nasal spray S212 (5, 10, 15 μL/mouse) could significantly increase the gait score of PD mice. And the nasal spray S212 (5, 10, 15 μL/mouse) had no obvious dose-dependent relationship, and its efficacy was similar to that of positive 100 mg/kg Medoba.

In summary, in the pole climbing test, 100 mg/kg Medoba and nasal spray S212 (5, 10, 15 μL/mouse) for 10 days of administration can improve the limb movement injury induced by MPTP in subacute PD model mice, showed as shortening the time of pole climbing and increasing the gait score of the mice. However, the nasal spray S212 (5, 10, 15 μL/mouse) had no obvious dose-dependent relationship, and were slightly weaker than the 100 mg/kg Medoba.

TABLE 2-1
The effect of S212 nasal spray via intranasal drug delivery
for 10 days (once a day) on the Time of climbing pole
in MPTP-induced subacute PD model mice(x ± sd)

Time of climbing pole (s)

| Group | N | Day 6 | Day 10 |
|---|---|---|---|
| Control | 10 | 6.79 ± 2.2 | 7.54 ± 2 |
| Model | 10 | 6.97 ± 2.39 | 14.68 ± 6.36## |
| Medopa (100 mg/kg) | 8 | 6.32 ± 2.37 | 4.43 ± 0.78*** |
| S212 (15 μL/mouse) | 10 | 5.21 ± 1.82 | 7.68 ± 3.22** |
| S212 (10 μL/mouse) | 10 | 5.22 ± 3.04 | 7.17 ± 3.65** |
| S212 (5 μL/mouse) | 10 | 5.26 ± 1.47 | 7.05 ± 1.81** |

##P < 0.01 vs. Control,
**P < 0.01,
***P < 0.001 vs. Model;
two mice of the Medopa group died.

TABLE 2-2
The effect of S212 nasal spray via intranasal drug delivery
for 10 days (once a day) on the Gait score of climbing
pole in MPTP-induced subacute PD model mice (x ± sd)

Gait score of climbing pole

| Group | N | Day 6 | Day 10 |
|---|---|---|---|
| Control | 10 | 4.67 ± 0.41 | 4.96 ± 0.11 |
| Model | 10 | 4.4 ± 0.49 | 3.77 ± 0.57### |
| Medopa (100 mg/kg) | 8 | 3.93 ± 0.32 | 4.5 ± 0.47** |
| S212 (15 μL/mouse) | 10 | 4.53 ± 0.32 | 4.47 ± 0.45** |
| S212 (10 μL/mouse) | 10 | 4.7 ± 0.43 | 4.6 ± 0.52** |
| S212 (5 μL/mouse) | 10 | 4.67 ± 0.38 | 4.3 ± 0.48* |

###P < 0.01 vs. Control,
*P < 0.05,
**P < 0.01 vs. Model;
two mice of the Medopa group died.

### The Effect of S212 Nasal Spray Via Intranasal Drug Delivery for 10 Days (Once a Day) on the Grip Strength in MPTP-Induced Subacute PD Model Mice

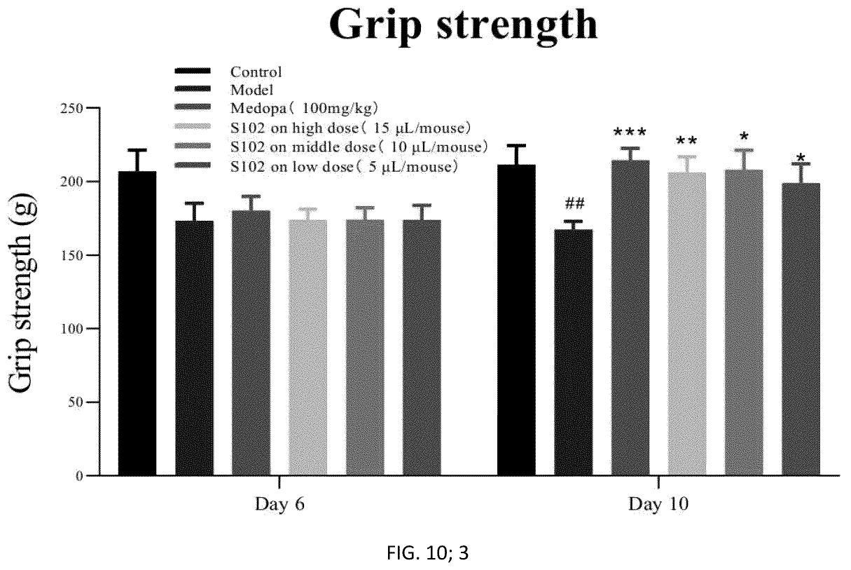

From the data in FIG. 10 (3) and Tab.3, the muscle tension and grip strength of mice in the model group decreased (P<0.01 vs. Control group). The 100 mg/kg Medoba significantly improved the grip strength of PD mice (P<0.001 vs Model group). The S212 (5, 10, 15 μL/mouse) significantly increased the grip strength of PD model mice, but there was no obvious dose-dependent relationship.

TABLE 3
The effect of S212 nasal spray via intranasal drug delivery
for 10 days (once a day) on the Gait score of climbing
pole in MPTP-induced subacute PD model mice(x ± sd)

| | | Gripping strength (g) | |
|---|---|---|---|
| Group | N | Day 6 | Day 10 |
| Control | 10 | 206.97 ± 45.72 | 211.42 ± 41.34 |
| Model | 10 | 173.29 ± 38.08 | 167.21 ± 17.31## |
| Medopa (100 mg/kg) | 8 | 180.19 ± 29.2 | 214.48 ± 22.57*** |
| S212 (15 μL/mouse) | 10 | 173.9 ± 23.22 | 206.03 ± 33.96* |
| S212 (10 μL/mouse) | 10 | 174.03 ± 25.62 | 207.91 ± 41.93* |
| S212 (5 μL/mouse) | 10 | 173.92 ± 31.01 | 198.88 ± 41.67* |

##P < 0.01 vs. Control,
*P < 0.05,
***P < 0.001 vs. Model;
two mice of the Medopa group died.

### The Effect of S212 Nasal Spray Via Intranasal Drug Delivery for 10 Days (Once a Day) on the Body Weight of the MPTP-Induced Subacute PD Model Mice

According to the data in Tab.4, during the administration period, the trend of weight changes in mice was relatively small. Compared with the Model group, the positive drug 100 mg/kg Medoba and test article S212 (5, 10, 15 μL/mouse) had no significant effect on the body weight of PD mice.

TABLE 4
The effect of S212 nasal spray via intranasal drug
delivery for 10 days (once a day) on the body weight
in MPTP-induced subacute PD model mice (x ± sd)

| | | Body weight (g) | | |
|---|---|---|---|---|
| Group | N | Day 1 | Day 6 | Day 10 |
| Control | 10 | 23.05 ± 1.35 | 24.22 ± 1.59 | 24.59 ± 1.45 |
| Model | 10 | 23.04 ± 0.71 | 23.66 ± 0.69 | 24.02 ± 0.95 |
| Medopa (100 mg/kg) | 8 | 23.51 ± 1.04 | 23.78 ± 1.14 | 24.1 ± 1.09 |
| S212 (15 μL/mouse) | 10 | 23.65 ± 0.96 | 24.11 ± 1.04 | 24.05 ± 1.09 |
| S212 (10 μL/mouse) | 10 | 23.39 ± 1.08 | 23.5 ± 1.07 | 23.87 ± 1.14 |
| S212 (5 μL/mouse) | 10 | 23.34 ± 1.41 | 23.97 ± 1.57 | 24.04 ± 1.31 |

Two mice of the Medopa group died.

### Conclusion

In this study, S212 nasal spray (5, 10, 15 μL/mouse) via intranasal drug delivery for 10 days (once a day) can improve the limb movement injury of subacute PD model mice induced by MPTP, but had no obvious dose-dependent relationship.

### Example 4—Evaluation of Pharmacokinetic Profiles of L-Carnosine after Single Nasal Drip Administration of S212 Nasal Spray in C57 Mice

The purpose of this study was to determine the pharmacokinetics profile of L-Carnosine by LC-MS/MS method in C57 mice following a single nasal drip administration. The analytical methods for Group 1 to Group 5 (G1-G5) were as follows:

### G1-G3 Analytical Method

| Instrument | LC-MS/MS-27 (TQ6500+) | |
|---|---|---|
| Matrix | C57 Mice Plasma | |
| Analyte(s) | L-Carnosine | |
| Internal | Wafarin (IS) | |
| standard(s) | | |
| MS | APCI: Positive | Q1/Q3 Masses: 227.30/156.20 Da |
| conditions | MRM detection | |
| | L-Carnosine | Q1/Q3 Masses: 309.10/163.10 Da |
| | IS: | |
| HPLC | Mobile phase: | |
| conditions | Mobile phase A: 2 mM Ammonium acetate in | |
| | 0.1% FA in water | |
| | Mobile phase B: 2 mM Ammonium formate and 0.1% FA in acetonitrile | |
| | weak wash: 50% MeOH/Water(v:v = 1:1) | |
| | strong wash: IPA:ACN:MeOH:0.1% FA in water(v:v:v:v, 1:1:1:1) | |
| | Time (min) | Moblie phase B (%) |
| | 0.01 | 90 |
| | 0.60 | 40 |
| | 1.20 | 40 |
| | 1.21 | 90 |
| | 1.50 | 90 |

| | |
|---|---|
| | Column: ACQUITY UPLC BEH Amide 1.7 um |
| | 2.1*50 mm |
| | Oven: 40° C. |
| | Flow rate: 0.60 mL/min |
| | Retention time: |
| | L-Carnosine: 1.04 min |
| | IS: 0.26 min |
| Sample | An aliquot of 12 μL plasma sample was protein precipitated with 240 μL MeOH in |
| preparation | which contains 10 ng/mL IS. The mixture was vortexed for 1 min and centrifuged at |
| | 18000 g for 7 min. Then for samples treated with tube were centrifuged at 14000 rpm for |
| | 7 min, but for samples treated with 96 well plates were centrifuged at 4000 rpm for 10 |
| | min. Transfer 220 μL supernatant to 96 well plates. An aliquot of 1 μL supernatant was |
| | injected for LC-MS/MS analysis. |
| Calibration | 20-20000 ng/mL for L-Carnosine in C57 Mice Plasma samples |
| curve | |

### G4 Analytical Method

| Instrument | LC-MS/MS-27 (TQ6500+) | |
|---|---|---|
| Matrix | C57 Mice Plasma | |
| Analyte(s) | L-Carnosine | |
| Internal | Wafarin (IS) | |
| standard(s) | | |
| MS | APCI: Positive | Q1/Q3 Masses: 227.30/156.20 Da |
| conditions | MRM detection | |
| | L-Carnosine | Q1/Q3 Masses: 309.10/163.10 Da |
| | IS: | |
| HPLC | Mobile phase: | |
| conditions | Mobile phase A: 2 mM Ammonium acetate in 0.1% FA in water | |
| | Mobile phase B: 2 mM Ammonium formate and 0.1% FA in acetonitrile | |
| | weak wash: 50% MeOH/Water(v:v = 1:1) | |
| | strong wash: IPA:ACN:MeOH:0.1% FA in water(v:v:v:v, 1:1:1:1) | |
| | Time (min) | Moblie phase B (%) |
| | 0.01 | 90 |
| | 0.60 | 40 |
| | 1.20 | 40 |
| | 1.21 | 90 |
| | 1.50 | 90 |

| | |
|---|---|
| | Column: ACQUITY UPLC BEH Amide 1.7 um |
| | 2.1*50 mm |
| | Oven: 40° C. |
| | Flow rate: 0.60 mL/min |
| | Retention time: |
| | L-Carnosine: 1.01 min |
| | IS: 0.26 min |
| Sample | An aliquot of 12 μL plasma sample was protein precipitated with 240 μL MeOH in |
| preparation | which contains 100 ng/mL IS. The mixture was vortexed for 1 min and centrifuged at |
| | 18000 g for 7 min. Then for samples treated with tube were centrifuged at 14000 rpm for |
| | 7 min, but for samples treated with 96 well plates were centrifuged at 4000 rpm for 10 |
| | min. Transfer 220 μL supernatant to 96 well plates. An aliquot of 1 μL supernatant was |
| | injected for LC-MS/MS analysis. |
| Calibration | 50-50000 ng/ml for L-Carnosine in C57 Mice Plasma samples |
| curve | |

### G5 Analytical Method

| Instrument | LC-MS/MS-27 (TQ6500+) | |
|---|---|---|
| Matrix | C57 Mice Plasma | |
| Analyte(s) | L-Carnosine | |
| Internal | Warfarin (IS) | |
| standard(s) | | |
| MS | APCI: Positive | Q1/Q3 Masses: 227.30/110.00 Da |
| conditions | MRM detection | |
| | L-Carnosine | Q1/Q3 Masses: 309.10/163.10 Da |
| | IS: | |
| HPLC | Mobile phase: | |
| conditions | Mobile phase A: 2 mM Ammonium acetate in 0.1% FA water | |
| | Mobile phase B: 2 mM Ammonium formate and 0.1% FA acetonitrile | |
| | weak wash: 50% MeOH/Water(v:v = 1:1) | |
| | strong wash: IPA:ACN:MeOH:0.1% FA in water(v:v:v:v, 1:1:1:1) | |
| | Time (min) | Moblie phase B (%) |
| | 0.01 | 90 |
| | 0.60 | 40 |
| | 1.20 | 40 |
| | 1.21 | 90 |
| | 1.50 | 90 |

| | |
|---|---|
| | Column: ACQUITY UPLC BEH Amide 1.7 um |
| | 2.1*50 mm |
| | Oven: 40° C. |
| | Flow rate: 0.60 mL/min |
| | Retention time: |
| | L-Carnosine: 1.05 min |
| | IS: 0.26 min |
| Sample | An aliquot of 12 μL plasma sample was protein precipitated with 240 μL MeOH in |
| preparation | which contains 100 ng/mL IS. The mixture was vortexed for 1 min and centrifuged at |
| | 18000 g for 7 min. Then for samples treated with tube were centrifuged at 14000 rpm for |
| | 7 min, but for samples treated with 96 well plates were centrifuged at 4000 rpm for 10 |
| | min. Transfer 220 μL supernatant to 96 well plates. An aliquot of 1 μL supernatant was |
| | injected for LC-MS/MS analysis. |
| Calibration | 50-50000 ng/mL for L-Carnosine in C57 Mice Plasma samples |
| curve | |

### Description of Test Articles

| | | Concentration | |
|---|---|---|---|
| Compound_ID | Batch NO. | (mg/mL) | PK NO. |
| S212 nasal | — | 200 | PK242757 |
| spray | | | |

### Description of Standard Articles

| | | Purity/ | | | |
|---|---|---|---|---|---|
| Compound_ID | Batch NO. | % | Molecular_Weight | Formula_Weight | PK NO. |
| L-Carnosine | C9625-10MG | ~99% | 226.23 | NA | PK242898 |

Note:
the test article is prepared as free form concentration. The purity does not need to adjustment.

### Test System

Species and Strain: C57BL/6J mice of SPF
Source: Medicilon Colony: 999M-018.
Gender: Male and Female
Number of Animals, transferred, 120 + 20 Male mouse, 20 Female mouse; used, transferred,
96 + 12 Male mouse, 12 Female mouse.
Selection for Study: No formal
randomization will be required.

### Study Design

| | | | | Dose | | Route | | |
|---|---|---|---|---|---|---|---|---|
| | | No. | Dose | Conc. | Dose | of | | |
| Group | TA | Male** | Level | (mg/mL) | Volume | Dosing | Collection | Analysis |
| 1 | saline | 24 | — | — | 10 | Nasal | Plasma | L-Carnosine |
| | | | | | μL/mice | drip, QD | | |
| 2 | S212 nasal | 24 | 1 | 200 | 5 | Nasal | | |
| | spray | | mg/mice | | μL/mice | drip, QD | | |
| 3 | S212 nasal | 24 | 2 | 200 | 10 | Nasal | | |
| | spray | | mg/mice | | μL/mice | drip, | | |
| | | | | | | BID, | | |
| | | | | | | interval | | |
| | | | | | | 6 h | | |
| 4 | S212 nasal | 24 | 3 | 200 | 15 | Nasal | | |
| | spray | | mg/mice | | μL/mice | drip, QD | | |

**Group1~Group4: n = 3/time/group.

| | | | Dose | | Dose | Route | | | |
|---|---|---|---|---|---|---|---|---|---|
| | | No.** | Level | Dose | Volume | of | | | |
| Group | TA | Male | Female | Conc. | (mg/mL) | Dosing | Collection | Analysis | |
| 5 | S212 nasal | 12 | 12 | 4 | 200 | 20 | Nasal | Plasma | L- |
| | spray | | | mg/mice | | μL/mice | drip, QD | | Carnosine |

**Group5: n = 6 (3 male mouse & 3 female mouse)/time/group.

### Rout of Administration

Group 1˜Group 5: The test articles was administered via single nasal drip administration. Group 4 and Group 5 were administered in two doses with a 4-minute interval between each dose. The blood collection time point was calculated from the second dose onwards.

### Collection Intervals

Group1 & Group2 & Group4: post-dose at 0.1, 0.5, 1, 2, 3, 4, 12, 24 h, total of 8 time points.

Group3: post-dose at 0.1, 0.5, 2, 6.1, 6.5, 8, 12, 24 h total of 8 time points.

Group5: pre-dose and post-dose at 2 min, 0.1 h, 0.5, 1, 2, 3, 4, 12, 24 h total of 10 time points.

### Blood Collection

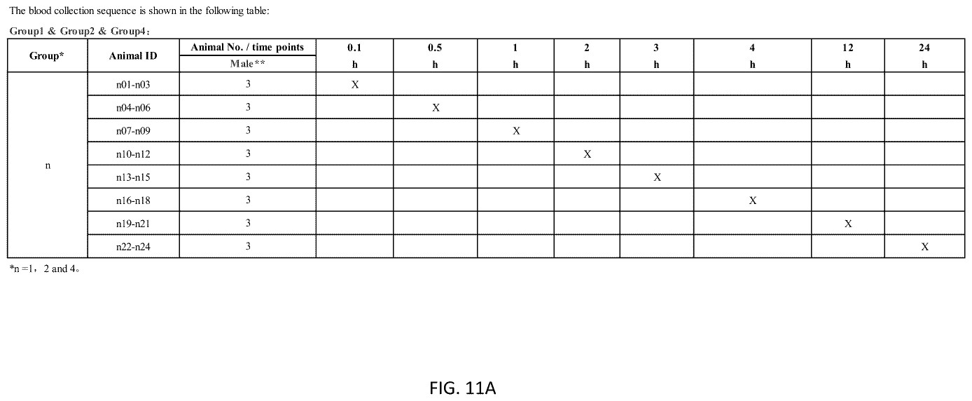

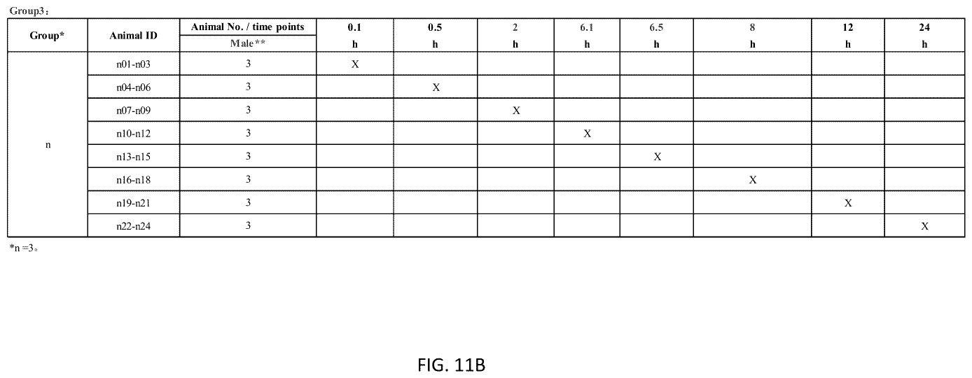

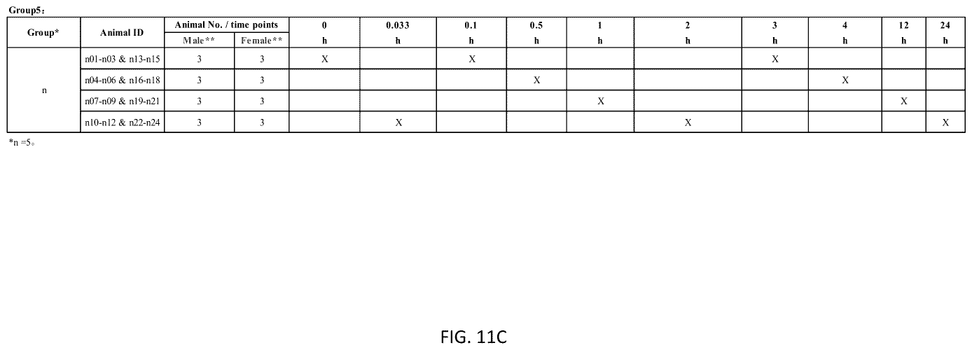

The blood collection sequence is shown in the table of FIGS. 11A, 11B, and 11C.

Blood Sample Collection and Procedure—The blood was taken via submandibular vein or other suitable vein, 0.03 mL/time point. Sample was placed in tubes containing K2-EDTA and stored on ice until centrifuged. The blood samples were centrifuged at 6800 g for 6 minutes at 2-8° C. within 1 h after collected and stored frozen at approximately −80° C.

Sample Analysis and Data Processing—The analytical result was confirmed using quality control samples for intra-assay variation. The accuracy of >66.7% of the quality control samples should be between 80-120% of the known value(s). Standard set of parameters including Area Under the Curve (AUC (0-t) and AUC (0-0)), elimination half-live (T½), maximum plasma concentration (Cmax), time to reach maximum plasma concentration (Tmax) were calculated using noncompartmental analysis modules in FDA certified pharmacokinetic program Phoenix WinNonlin 7.0 (Pharsight, USA) by the Study Director.

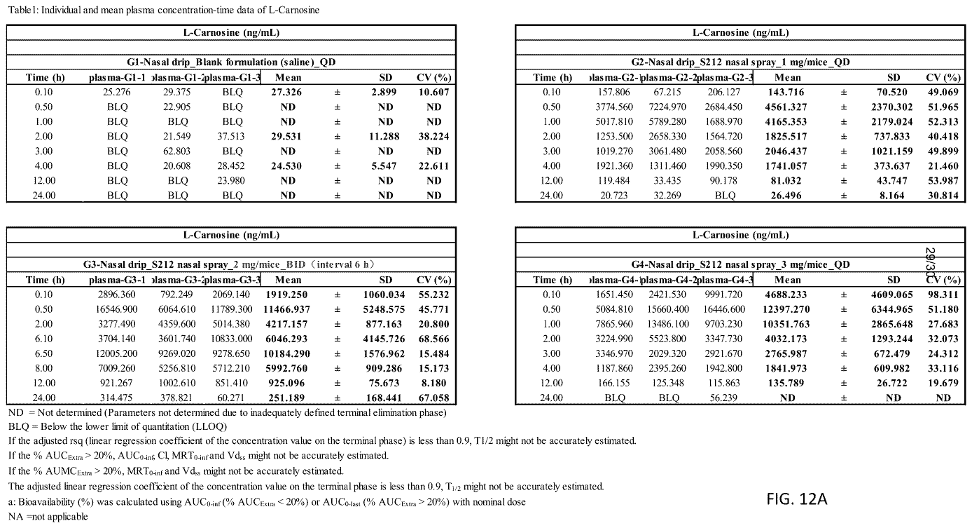

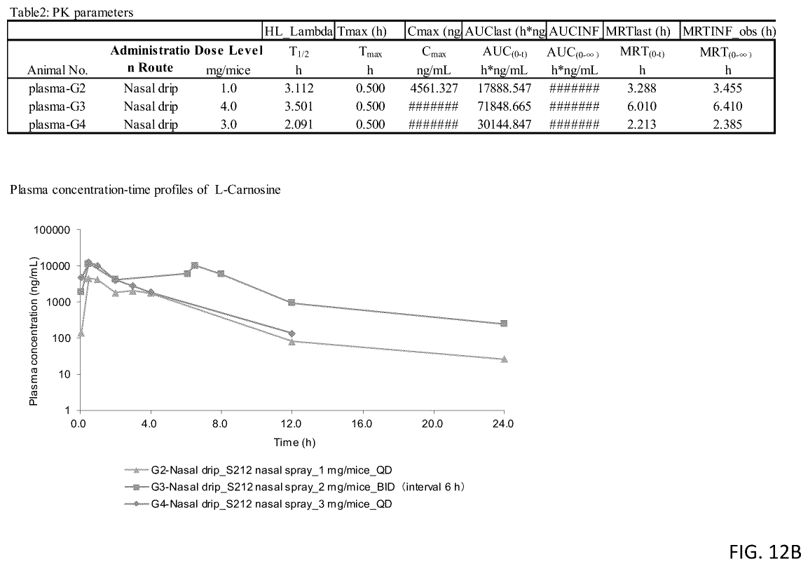

Results of the study are show in FIGS. 12A and 12B. As is apparent, significant levels of L-Carnosine is observed at all time points following S212 administration that is sustained for at least 24 hrs.

All documents cited in this application are hereby incorporated by reference as if recited in full herein. In the event of a conflict between the teachings of this application and those of the incorporated documents, the teachings of this application control.

The embodiments described in this disclosure can be combined in various ways. Any aspect or feature that is described for one embodiment can be incorporated into any other embodiment mentioned in this disclosure. While various novel features of the inventive principles have been shown, described and pointed out as applied to particular embodiments thereof, it should be understood that various omissions and substitutions and changes can be made by those skilled in the art without departing from the spirit of this disclosure. Those skilled in the art will appreciate that the inventive principles can be practiced in other than the described embodiments, which are presented for purposes of illustration and not limitation.

What is claimed is:

1. An intranasal spray composition comprising:
a peptide
a thickening agent;
a cyclodextrin (CD); and
a surfactant;
wherein the peptide and cyclodextrin form an inclusion complex and are encapsulated by the surfactant;
wherein the peptide, CMC, CD, and non-ionic surfactant are effective to produce a stable population of particles.

2. The composition of claim 1, wherein the thickening agent is selected from the group consisting of methylcellulose, ethylcellulose, hydroxy-ethylcellulose, hydroxyl propyl cellulose, hydroxy propyl methylcellulose, carboxymethyl cellulose, sodium carboxy methylcellulose; polyacrylic acid polymers, poly hydroxyethyl methylacrylate; polyethylene oxide; polyvinyl pyrrolidone; polyvinyl alcohol, tragacanth, sodium alginate, araya gum, guar gum, xanthan gum. lectin, soluble starch, gelatin, pectin and chitosan.

3. The composition of claim 1, wherein the thickening agent is a cellulose.

4. The composition of claim 1, wherein the thickening agent is a carboxymethyl cellulose (CMC).

5. The composition of claim 1, wherein the surfactant is a non-ionic surfactant.

6. The composition of claim 1, wherein the surfactant is selected from the group consisting of Polysorbate 80, Polysorbate 20, peg 40 hydrogenated castor oil. Surfactants also include but are not limited to: Polysorbate 80 NF, polyoxyethylene 20 sorbitan monolaurate, polyoxyethylene (4) sorbitan monolaurate, polyoxyethylene 20 sorbitan monopalmitate, polyoxyethylene 20 sorbitan monostearate, polyoxyethylene (4) sorbitan monostearate, polyoxyethylene 20 sorbitan tristearate, polyoxyethylene (5) sorbitan monooleate, polyoxyethylene 20 sorbitan trioleate, polyoxyethylene 20 sorbitan monoisostearate, sorbitan monooleate, sorbitan monolaurate, sorbitan monopalmitate, sorbitan monostearate, sorbitan trilaurate, sorbitan trioleate, sorbitan tristearate, and any combination thereof.

7. The composition of claim 1, wherein the surfactant is polysorbate 80, polysorbate 20, peg 40, hydrogenated castor oil, and combinations thereof.

8. The composition of claim 1, wherein the surfactant is Polysorbate 80.

9. The composition of claim 1, wherein the cyclodextrin is selected from the group consisting of alpha cyclodextrin, beta cyclodextrin, a gamma cyclodextrin, a 2-hydroxypropyl-β-cyclodextrin, a β-cyclodextrin sulfobutylether, hydroxyethyl-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-1-β-cyclodextrin, glycosyl-β-cyclodextrin, maltosyl-β-cyclodextrin, and any combination thereof.

10. The composition of claim 1, wherein the cyclodextrin is a gamma cyclodextrin.

11. The composition of claim 1, wherein the peptide and cyclodextrin are encapsulated by the surfactant as a liposome, micelle, or noisome.

12. The composition of claim 1, comprising 1%-40% (wt) peptide, 0.1%-5% (wt) thickening agent, 1%-15% (wt) CD, and 0.010%-0.025% surfactant.

13. The composition of claim 1, wherein the surfactant is selected from the group consisting of polysorbate 80, polysorbate 20, peg 40 hydrogenated castor oil, and combinations thereof.

14. The composition of claim 1, further comprising one or more of a preservative and a chelator.

15. The composition of claim 1, further comprising one or more of potassium sorbate and EDTA.

16. The composition of claim 1, wherein the composition has a pH of 8.25 and 8.31.

17. The composition of claim 1, wherein 90% of the particles are 50 μm or less in diameter.

18. The composition of claim 1, wherein 10% of the particles are 15 μm or less in diameter.

19. The composition of claim 1, wherein 50% of the particles are 25 μm or less in diameter.

20. A method of treating a subject with a therapeutic formulation comprising the step of intranasally administering a composition comprising:
a peptide
a thickening agent;
a cyclodextrin (CD); and
a surfactant;
wherein the peptide and cyclodextrin form an inclusion complex and are encapsulated by the surfactant;
wherein the peptide, CMC, CD, and non-ionic surfactant are effective to produce a stable population of particles.

21. The method of claim 20, wherein the thickening agent is selected from the group consisting of methylcellulose, ethylcellulose, hydroxy-ethylcellulose, hydroxyl propyl cellulose, hydroxy propyl methylcellulose, carboxymethyl cellulose, sodium carboxy methylcellulose; polyacrylic acid polymers, poly hydroxyethyl methylacrylate; polyethylene oxide; polyvinyl pyrrolidone; polyvinyl alcohol, tragacanth, sodium alginate, araya gum, guar gum, xanthan gum. lectin, soluble starch, gelatin, pectin and chitosan.

22. The method of claim 20, wherein the thickening agent is a carboxymethyl cellulose (CMC).

23. The method of claim 20, wherein the surfactant is a non-ionic surfactant.

24. The method of claim 20, wherein the surfactant is selected from the group consisting of Polysorbate 80, Polysorbate 20, peg 40 hydrogenated castor oil. Surfactants also include but are not limited to: Polysorbate 80 NF, polyoxyethylene 20 sorbitan monolaurate, polyoxyethylene (4) sorbitan monolaurate, polyoxyethylene 20 sorbitan monopalmitate, polyoxyethylene 20 sorbitan monostearate, polyoxyethylene (4) sorbitan monostearate, polyoxyethylene 20 sorbitan tristearate, polyoxyethylene (5) sorbitan monooleate, polyoxyethylene 20 sorbitan trioleate, polyoxyethylene 20 sorbitan monoisostearate, sorbitan monooleate, sorbitan monolaurate, sorbitan monopalmitate, sorbitan monostearate, sorbitan trilaurate, sorbitan trioleate, sorbitan tristearate, and any combination thereof.

25. The method of claim 20, wherein the surfactant is polysorbate 80, polysorbate 20, peg 40, hydrogenated castor oil, and combinations thereof

26. The method of claim 20, wherein the surfactant is Polysorbate 80.

27. The method of claim 20, wherein the cyclodextrin is selected from the group consisting of alpha cyclodextrin, beta cyclodextrin, a gamma cyclodextrin, a 2-hydroxypropyl-β-cyclodextrin, a β-cyclodextrin sulfobutylether, hydroxyethyl-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-1-β-cyclodextrin, glycosyl-β-cyclodextrin, maltosyl-β-cyclodextrin, and any combination thereof.

28. The method of claim 20, wherein the cyclodextrin is a gamma cyclodextrin.

29. The method of claim 20, wherein the peptide and cyclodextrin are encapsulated by the surfactant as a liposome, micelle, or noisome.

30. The method of claim 20, comprising 1%-40% (wt) peptide, 0.1%-5% (wt) thickening agent, 1%-15% (wt) CD, and 0.010%-0.025% surfactant.

31. The method of claim 20, wherein the surfactant is selected from the group consisting of polysorbate 80, polysorbate 20, peg 40 hydrogenated castor oil, and combinations thereof.

32. The method of claim 20, further comprising one or more of a preservative and a chelator.

33. The method of claim 20, further comprising one or more of potassium sorbate and EDTA.

34. The method of claim 20, wherein the composition has a pH of 8.25 and 8.31.

35. The method of claim 20, wherein 90% of the particles are 50 μm or less in diameter.

36. The method of claim 20, wherein 10% of the particles are 15 μm or less in diameter.

37. The method of claim 20, wherein 50% of the particles are 25 μm or less in diameter.

38. A method for delivery of a peptide to the central nervous system of a subject by intranasal administration comprising the step of:
providing to the subject a compositions comprising a peptide;
a thickening agent;
a cyclodextrin (CD); and
a surfactant;
wherein the peptide and cyclodextrin form an inclusion complex and are encapsulated by the surfactant;
wherein the peptide, CMC, CD, and non-ionic surfactant are effective to produce a stable population of particles.

39. The method of claim 38, wherein the composition is administered intranasally 2 or more times per day.

40. The method of claim 38, wherein the thickening agent is selected from the group consisting of methylcellulose, ethylcellulose, hydroxy-ethylcellulose, hydroxyl propyl cellulose, hydroxy propyl methylcellulose, carboxymethyl cellulose, sodium carboxy methylcellulose; polyacrylic acid polymers, poly hydroxyethyl methylacrylate; polyethylene oxide; polyvinyl pyrrolidone; polyvinyl alcohol, tragacanth, sodium alginate, araya gum, guar gum, xanthan gum. lectin, soluble starch, gelatin, pectin and chitosan.

41. The method of claim 38, wherein the thickening agent is a carboxymethyl cellulose (CMC).

42. The method of claim 38, wherein the surfactant is a non-ionic surfactant.

43. The method of claim 38, wherein the surfactant is selected from the group consisting of Polysorbate 80, Polysorbate 20, peg 40 hydrogenated castor oil. Surfactants also include but are not limited to: Polysorbate 80 NF, polyoxyethylene 20 sorbitan monolaurate, polyoxyethylene (4) sorbitan monolaurate, polyoxyethylene 20 sorbitan monopalmitate, polyoxyethylene 20 sorbitan monostearate, polyoxyethylene (4) sorbitan monostearate, polyoxyethylene 20 sorbitan tristearate, polyoxyethylene (5) sorbitan monooleate, polyoxyethylene 20 sorbitan trioleate, polyoxyethylene 20 sorbitan monoisostearate, sorbitan monooleate, sorbitan monolaurate, sorbitan monopalmitate, sorbitan monostearate, sorbitan trilaurate, sorbitan trioleate, sorbitan tristearate, and any combination thereof.

44. The method of claim 38, wherein the surfactant is polysorbate 80, polysorbate 20, peg 40, hydrogenated castor oil, and combinations thereof

45. The method of claim 38, wherein the surfactant is Polysorbate 80.

46. The method of claim 38, wherein the cyclodextrin is selected from the group consisting of alpha cyclodextrin, beta cyclodextrin, a gamma cyclodextrin, a 2-hydroxypropyl-β-cyclodextrin, a β-cyclodextrin sulfobutylether, hydroxyethyl-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-1-β-cyclodextrin, glycosyl-β-cyclodextrin, maltosyl-β-cyclodextrin, and any combination thereof.

47. The method of claim 38, wherein the cyclodextrin is a gamma cyclodextrin.

48. The method of claim 38, wherein the peptide and cyclodextrin are encapsulated by the surfactant as a liposome, micelle, or noisome.

49. The method of claim 38, comprising 1%-40% (wt) peptide, 0.1%-5% (wt) thickening agent, 1%-15% (wt) CD, and 0.010%-0.025% surfactant.

50. The method of claim 38, wherein the surfactant is selected from the group consisting of polysorbate 80, polysorbate 20, peg 40 hydrogenated castor oil, and combinations thereof.

51. The method of claim 38, further comprising one or more of a preservative and a chelator.

52. The method of claim 38, further comprising one or more of potassium sorbate and EDTA.

53. The method of claim 38, wherein the composition has a pH of 8.25 and 8.31.

54. The method of claim 38, wherein 90% of the particles are 50 μm or less in diameter.

55. The method of claim 38, wherein 10% of the particles are 15 μm or less in diameter.

56. The method of claim 38, wherein 50% of the particles are 25 μm or less in diameter.
